(19) United States
(12) Patent Application Publication
(10) Pub. No.: US 2026/0347856 A1
(43) Pub. Date: Nov. 5, 2026

(54) COMPOSITIONS AND METHODS FOR TREATMENT OF NEUROINFLAMMATORY DISEASES

(71) Applicant: Atalanta Therapeutics, Inc., Boston, MA (US)

(72) Inventors: Corrie GALLANT-BEHM, Wellesley, MA (US); Wimalanathan KOKULAPALAN, Cambridge, MA (US); Benjamin ANDREONE, Boston, MA (US); Matthew HASSLER, Boston, MA (US); Daniel CURTIS, Belmont, MA (US); Bruno Miguel DA CRUZ GODINHO, Reading, MA (US)

(21) Appl. No.: 19/115,146

(22) PCT Filed: Sep. 28, 2023
(86) PCT No.: PCT/US2023/075406

§ 371 Date: Mar. 25, 2025

Related U.S. Application Data
(60) Provisional application No. 63/410,872, filed on Sep. 28, 2022.

(51) Int. Cl. C12N 15/113; A61K 31/712; A61K 31/7125

(52) U.S. Cl. CPC C12N 15/1138; A61K 31/712; A61K 31/7125; C12N 2310/14; C12N 2310/315; C12N 2310/321; C12N 2310/322; C12N 2310/3515; C12N 2310/52

(57) ABSTRACT

The present disclosure provides single- or double-stranded interfering RNA molecules (e.g., siRNA) that target a membrane-spanning 4-domains AGA (MS4A6A) gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a neuroinflammatory disease (e.g., Alzheimer's disease).

### SEQUENCE LISTING

The instant application contains a Sequence Listing which has been submitted electronically in XML format and is hereby incorporated by reference in its entirety. Said XML copy, created on Sep. 19, 2023, is named “51436-036WO2_Sequence_Listing_9_19_23” and is 505,859 bytes in size.

### TECHNICAL FIELD

This disclosure relates to small interfering RNA (siRNA) molecules, and compositions containing the same, that target RNA transcripts (e.g., mRNA) of a membrane-spanning 4-domains subfamily A (MS4A) gene (e.g., MS4A6A). The disclosure further describes methods for silencing of MS4A6A and the treatment of diseases that may benefit from the silencing of MS4A6A (e.g., neuroinflammatory diseases such as Alzheimer's disease) by delivering MS4A6A-targeting siRNA molecules to a target tissue of a subject in need.

### BACKGROUND

The MS4A (membrane-spanning 4-domains subfamily A) gene family encodes proteins that are implicated in the pathology of diseases including neuroinflammatory diseases (e.g., Alzheimer's disease). Accordingly, there is a need for therapeutics capable of selectively diminishing MS4A activity (e.g., MS4A6A) in a manner that provides effective treatment for neuroinflammatory diseases (e.g., Alzheimer's disease) or other MS4A-related diseases or disorders.

### SUMMARY OF THE INVENTION

The present disclosure provides compositions and methods for reduction of membrane-spanning 4-domains subfamily A (MS4A) expression by way of small interfering RNA (siRNA)-mediated silencing of MS4A transcripts, specifically MS4A6A. The compositions and methods provide the benefit of exhibiting high selectivity toward MS4A6A over other genes (e.g., other MS4A family members).

The siRNA molecules of the disclosure can be used to silence the MS4A6A gene, thereby preventing the translation of the corresponding mRNA transcript and reducing membrane-spanning 4-domains A6A protein expression. This reduction of membrane-spanning 4-domains A6A levels thus prevents disease onset or progression, as variants of the MS4A6A gene has been associated with the onset of neuroinflammatory disease (e.g., Alzheimer's disease). The siRNA molecules of the disclosure can be delivered directly to a subject in need of MS4A6A silencing by way of, for example, injection intrathecally, intracerebroventricularly, intrastriatally, intraparenchymally, intra-cisterna magna injection, such as by catheterization, intravenous injection, subcutaneous injection, or intramuscular injection.

In an aspect, the disclosure provides an siRNA molecule containing an antisense strand and sense strand having complementarity to the antisense strand. The antisense strand has complementarity sufficient to hybridize to a region within a MS4A6A mRNA transcript having the nucleic acid sequence of any one of SEQ ID NOs: 1-192. The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length). In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments of any of the foregoing aspects, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 15 contiguous nucleobases within the MS4A6A mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 16 contiguous nucleobases within the MS4A6A mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 17 contiguous nucleobases within the MS4A6A mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 18 contiguous nucleobases within the MS4A6A mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 19 contiguous nucleobases within the MS4A6A mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 20 contiguous nucleobases within the MS4A6A mRNA transcript having the nucleic acid sequence of any one of SEQ ID NOs: 1-192. In some embodiments, the antisense strand has at least 70% (e.g., at least 70%, at least 71%, at least 72%, at least 73%, at least 74%, at least 75%, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or 100%) complementarity to a region of 21 contiguous nucleobases within the MS4A6A mRNA transcript having the nucleic acid sequence of any one of SEQ ID NOs: 1-192.

In some embodiments, the antisense strand has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to the region within the MS4A6A mRNA transcript having the nucleic acid sequence of any one of SEQ ID NOs: 1-192.

In some embodiments, the antisense strand has at least 75% complementarity to the region within the MS4A6A mRNA transcript having the nucleic acid sequence of any one of SEQ ID NOs: 1-192. For example, the antisense strand may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the MS4A6A mRNA transcript having the nucleic acid sequence of any one of SEQ ID NOs: 1-192.

In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the MS4A6A mRNA transcript having the nucleic acid sequence of any one of SEQ ID NOs: 1-192.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the MS4A6A mRNA transcript having the nucleic acid sequence of any one of SEQ ID NOs: 1-192.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the MS4A6A mRNA transcript having the nucleic acid sequence of any one of SEQ ID NOs: 1-192.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the MS4A6A mRNA transcript having the nucleic acid sequence of any one of SEQ ID NOs: 1-192.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the MS4A6A RNA transcript having the nucleic acid sequence of any one of SEQ ID NOs: 1-192.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the MS4A6A mRNA transcript having the nucleic acid sequence of any one of SEQ ID Nos: 1-192.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the MS4A6A mRNA transcript having the nucleic acid sequence of any one of SEQ ID NOs: 1-192.

In some embodiments, the antisense strand has 21 contiguous nucleotides that are fully complementary to a contiguous polynucleotide segment of equal length within the region of the MS4A6A mRNA transcript having the nucleic acid sequence of any one of SEQ ID NOs: 1-192.

In some embodiments, the antisense strand has 9 or fewer nucleotide mismatches relative to a region of 21 contiguous nucleobases of the MS4A6A RNA transcript having the nucleic acid sequence of any one of SEQ ID NOs: 1-192, optionally wherein the antisense strand contains 8 or fewer, 7 or fewer, 6 or fewer, 5 or fewer, 4 or fewer, 3 or fewer, 2 or fewer, or only 1 mismatch relative to the region of the MS4A6A mRNA transcript having the nucleic acid sequence of any one of SEQ ID NOs: 1-192.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 385-576.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 385-576.

In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 385-576, optionally wherein the antisense strand has a nucleic acid sequence that is at least 96%, 97%, 98%, or 99% identical to the nucleic acid sequence of any one of SEQ ID NOs: 385-576.

In some embodiments, the antisense strand has the nucleic acid sequence of any one of SEQ ID NOs: 385-576.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 193-384.

In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 193-384, optionally wherein the sense strand has a nucleic acid sequence that is at least 96%, 97%, 98%, or 99% identical to the nucleic acid sequence of any one of SEQ ID NOs: 193-384.

In some embodiments, the siRNA molecule has a sense strand having the nucleic acid sequence of any one of SEQ ID NOs: 193-384.

In some embodiments, the antisense strand has a structure represented by Formula I, wherein Formula I is, in the 5′-to-3′ direction:

A-B-(A′)ⱼ-C-P²-D-P¹—(C′-P¹)ₖ-C′ Formula I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

A-B-(A′)ⱼ-C-P²-D-P¹—(C-P¹)ₖ-C′ Formula II;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

E-(A′)ₘ-F Formula III;
wherein E is represented by the formula (C-P¹)₂;
F is represented by the formula (C-P²)₃-D-P¹-C-P¹—C, (C-P²)₃-D-P²-C-P²—C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

A-(A′)ⱼ-C-P²—B—(C-P¹)ₖ-C′ Formula IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

E-(A′)ₘ-C-P²—F Formula V;
wherein E is represented by the formula (C-P¹)₂;
F is represented by the formula D-P¹-C-P¹—C, D-P²-C-P²—C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

A-Bⱼ-E-Bₖ-E-F-Gₗ-D-P¹—C′ Formula VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

H-Bₘ-Iₙ-A′-Bₒ-H-C Formula VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
o is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

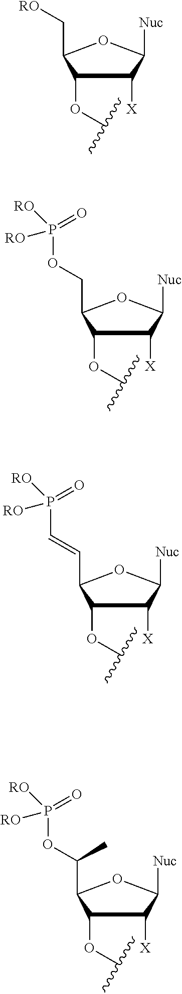

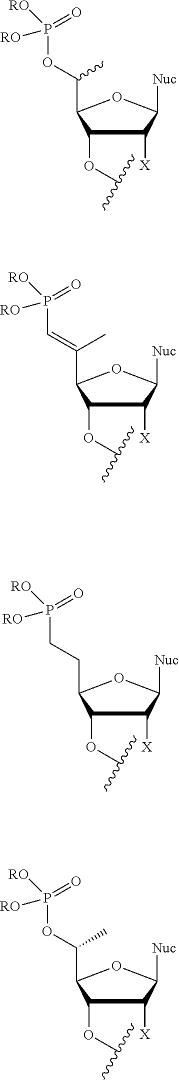

wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, and tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

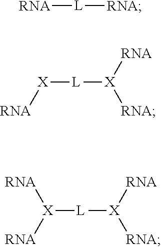

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

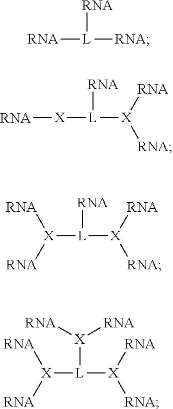

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched siRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

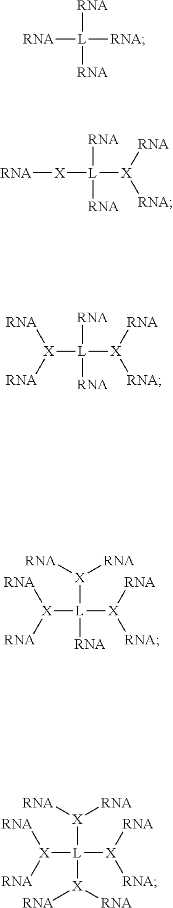

wherein each RNA is, independently, an siRNA molecule, L is a linker, and each X, independently, represents a branch point moiety.

In some embodiments, the tetra-branched siRNA molecule is represented by Formula XXIV. In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

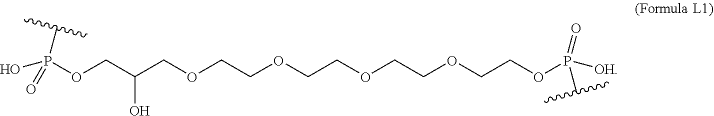 [SMILES: *P(=O)(O)OCCOCCOCCOCCOCC(O)COP(*)(=O)O]

In some embodiments, the linker includes a structure of Formula L2:

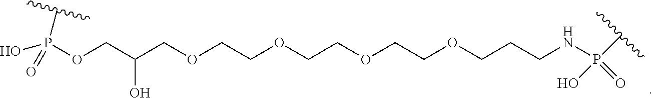 [SMILES: *P(=O)(O)NCCCOCCOCCOCCOCC(O)COP(*)(=O)O]

In some embodiments, the linker includes a structure of Formula L3:

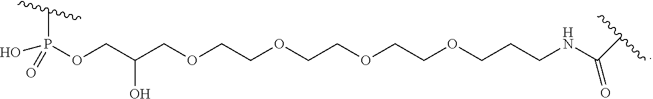 [SMILES: *C(=O)NCCCOCCOCCOCCOCC(O)COP(*)(=O)O]

In some embodiments, the linker includes a structure of Formula L4:

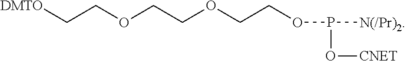

In some embodiments, the linker includes a structure of Formula L5:

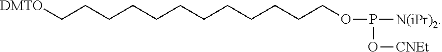

In some embodiments, the linker includes a structure of Formula L6:

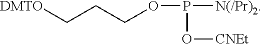

In some embodiments, the linker includes a structure of Formula L7:

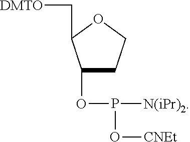

In some embodiments, the linker includes a structure of Formula L8:

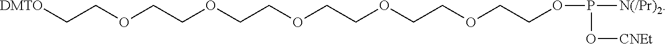

In some embodiments, the linker includes a structure of Formula L9:

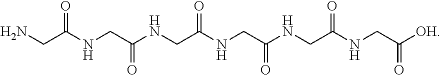 [SMILES: NCC(=O)NCC(=O)NCC(=O)NCC(=O)NCC(=O)NCC(=O)O]

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides.

In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to a subject diagnosed as having a neuroinflammatory disease by administering a therapeutically effective amount of the siRNA molecule or a pharmaceutical composition of any of the preceding aspects or embodiments of the disclosure to the subject.

In a further aspect, the disclosure provides a method of treating a neuroinflammatory disease in a subject in need thereof by administering a therapeutically effective amount of an siRNA molecule or a pharmaceutical composition of any of the preceding aspects or embodiments of the disclosure to the subject.

In some embodiments of the methods described herein, the neuroinflammatory disease is Alzheimer's disease.

In another aspect, the disclosure provides a method of reducing MS4A6A expression in a subject in need thereof by administering a therapeutically effective amount of an siRNA or pharmaceutical composition of any of the preceding aspects or embodiments of the disclosure.

In some embodiments of the methods described herein, the siRNA molecule or the pharmaceutical composition is administered to the subject by way of intracerebroventricular, intrastriatal, intraparenchymal or intrathecal injection. In some embodiments, the siRNA molecule or the pharmaceutical composition is administered to the subject by way of intravenous, intramuscular, or subcutaneous injection.

In some embodiments of any of the methods described herein, the subject is a human.

In another aspect, the disclosure provides a kit containing an siRNA molecule or pharmaceutical composition of any of the preceding aspects or embodiments of the disclosure, and a package insert that instructs a user of the kit to perform the method of any of the preceding aspects or embodiments of the disclosure.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition.

Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems.

Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)
where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)
where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5 °C less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of MS4A6A, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂.

In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C═C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C═CH and —C═C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl group generally has the formula of phenyl-CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human), that experiences a neuroinflammatory disease or disorder (e.g., Alzheimer's disease) and/or contains a gain-of-function MS4A6A variant allele or a polymorphism in the MS4A6A gene that is linked to disease pathology.

As used herein, the term “MS4A6A” refers to the gene encoding membrane-spanning 4-domains A6A, including any native MS4A6A gene from any source. The term encompasses “full-length,” unprocessed MS4A6A as well as any form of MS4A6A that results from processing in the cell. The term also encompasses naturally occurring variants of MS4A6A, e.g., splice variants or allelic variants. The nucleic acid sequence of an exemplary MS4A6A gene is shown in European Nucleotide Archive (ENA) Accession No. AF237908.1. The amino acid sequence of an exemplary protein encoded by an MS4A6A gene is shown in UNIPROT™ Accession No. Q9H2W1.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on pharmacological treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical, cognitive, or behavioral (e.g., depressive behavior or apathy) parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, a neuroinflammatory disease such as Alzheimer's disease. For example, clinical benefits in the context of a subject having Alzheimer's disease administered an siRNA molecule or siRNA composition of the disclosure include, without limitation, a reduction in cognitive impairment or memory loss. “Benefit” and “response” are also used interchangeably to refer to, for example, a reduction in wild type MS4A6A transcripts, mutant MS4A6A transcripts, variant MS4A6A transcripts, splice isoforms of MS4A6A transcripts, and/or overexpressed MS4A6A transcripts.

### DETAILED DESCRIPTION

The present disclosure provides compositions of small interfering RNA (siRNA) molecules with sequence homology to a membrane-spanning 4-domains A6A (MS4A6A) gene and methods for administering said siRNA molecules to a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference (RNAi) by degrading mRNA with a complementary nucleotide sequence, thus preventing the translation of the target gene.

The siRNA molecules of the disclosure may exhibit, for example, robust gene-specific suppression of MS4A6A, relative to other human genes.

The siRNA molecules of the disclosure may feature an antisense strand having a nucleic acid sequence that is complementary to a region of a MS4A6A mRNA transcript having the nucleic acid sequence of any one of SEQ ID NOs: 1-192. The degree of complementarity of the antisense strand to the region of the MS4A6A mRNA transcript may be sufficient for the antisense strand to anneal over the full length of the region of the MS4A6A mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the MS4A6A mRNA transcript.

In some embodiments, the siRNA molecules of the disclosure feature an antisense strand having the nucleic acid sequence of any one of SEQ ID NOs: 385-576, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 385-576.

In some embodiments, the siRNA molecules of the disclosure feature a sense strand having the nucleic acid sequence of any one of SEQ ID NOs: 193-384, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 193-384.

Exemplary siRNA molecules of the disclosure are those shown in Table 1, below. Table 1 summarizes the antisense strands, sense strands, and corresponding regions of a MS4A6A mRNA transcript that are targeted by each antisense strand.

TABLE 1
Nucleotide sequences for gene-specific MS4A6A-targeting siRNA

| Gene | | | | | |
|---|---|---|---|---|---|
| region | | Sense | | Antisense | |
| SEQ | | SEQ | | SEQ | |
| ID | | ID | | ID | |
| NO: | Gene Region | NO: | Sense Sequence | NO: | Antisense Sequence |
| 1 | GAUGACUUUCA | 193 | CUUUCAGAAGAU | 385 | UUUCAUCUUCUGAAA |
| | GAAGAUGAAG | | GAAA | | GUCAUC |
| 2 | AUGACUUUCAG | 194 | UUUCAGAAGAUG | 386 | UCUUCAUCUUCUGAA |
| | AAGAUGAAGG | | AAGA | | AGUCAU |
| 3 | UGACUUUCAGA | 195 | UUCAGAAGAUGAA | 387 | UCCUUCAUCUUCUGA |
| | AGAUGAAGGU | | GGA | | AAGUCA |
| 4 | UUCAGAAGAUG | 196 | AAGAUGAAGGUAA | 388 | UACUUACCUUCAUCU |
| | AAGGUAAGUA | | GUA | | UCUGAA |
| 5 | UCAGAAGAUGAA | 197 | AGAUGAAGGUAA | 389 | UUACUUACCUUCAUC |
| | GGUAAGUAG | | GUAA | | UUCUGA |
| 6 | CAGAAGAUGAA | 198 | GAUGAAGGUAAG | 390 | UCUACUUACCUUCAU |
| | GGUAAGUAGA | | UAGA | | CUUCUG |
| 7 | AGAAGAUGAAG | 199 | AUGAAGGUAAGU | 391 | UUCUACUUACCUUCA |
| | GUAAGUAGAA | | AGAA | | UCUUCU |
| 8 | GAAGAUGAAGG | 200 | UGAAGGUAAGUA | 392 | UUUCUACUUACCUUC |
| | UAAGUAGAAA | | GAAA | | AUCUUC |
| 9 | GAUGAAGGUAA | 20 | AGGUAAGUAGAAA | 393 | UGGUUUCUACUUACC |
| | GUAGAAACCG | | CCA | | UUCAUC |
| 10 | ACCGUUGAUGG | 202 | UGAUGGGACUGA | 394 | UUUCUCAGUCCCAUC |
| | GACUGAGAAA | | GAAA | | AACGGU |
| 11 | GAAACCAGAGU | 203 | CAGAGUUAAAACC | 395 | UGAGGUUUUAACUCU |
| | UAAAACCUCU | | UCA | | GGUUUC |
| 12 | AACCAGAGUUAA | 204 | GAGUUAAAACCUC | 396 | UAAGAGGUUUUAACU |
| | AACCUCUUU | | UUA | | CUGGUU |
| 13 | UUUGGAGCUUC | 205 | AGCUUCUGAGGA | 397 | UGAGUCCUCAGAAGC |
| | UGAGGACUCA | | CUCA | | UCCAAA |
| 14 | UUGGAGCUUCU | 206 | GCUUCUGAGGAC | 398 | UUGAGUCCUCAGAAG |
| | GAGGACUCAG | | UCAA | | CUCCAA |
| 15 | CGGGCACAGUU | 207 | ACAGUUGGCAAC | 399 | UGGUGUUGCCAACUG |
| | GGCAACACCA | | ACCA | | UGCCCG |
| 16 | GGCACAGUUGG | 208 | AGUUGGCAACAC | 400 | UAUGGUGUUGCCAAC |
| | CAACACCAUC | | CAUA | | UGUGCC |
| 17 | GCACAGUUGGC | 209 | GUUGGCAACACC | 401 | UGAUGGUGUUGCCAA |
| | AACACCAUCA | | AUCA | | CUGUGC |
| 18 | CACAGUUGGCA | 210 | UUGGCAACACCA | 402 | UUGAUGGUGUUGCCA |
| | ACACCAUCAU | | UCAA | | ACUGUG |
| 19 | AGUUGGCAACA | 211 | GCAACACCAUCAU | 403 | UUCAUGAUGGUGUUG |
| | CCAUCAUGAC | | GAA | | CCAACU |
| 20 | GUUGGCAACAC | 212 | CAACACCAUCAUG | 404 | UGUCAUGAUGGUGUU |
| | CAUCAUGACA | | ACA | | GCCAAC |
| 21 | CAACACCAUCAU | 213 | CCAUCAUGACAUC | 405 | UGUGAUGUCAUGAUG |
| | GACAUCACA | | ACA | | GUGUUG |
| 22 | AACACCAUCAUG | 214 | CAUCAUGACAUCA | 406 | UUGUGAUGUCAUGAU |
| | ACAUCACAA | | CAA | | GGUGUU |
| 23 | ACACCAUCAUGA | 215 | AUCAUGACAUCAC | 407 | UUUGUGAUGUCAUGA |
| | CAUCACAAC | | AAA | | UGGUGU |
| 24 | UCAUGACAUCAC | 216 | ACAUCACAACCUG | 408 | UAACAGGUUGUGAUG |
| | AACCUGUUC | | UUA | | UCAUGA |
| 25 | CAUGACAUCACA | 217 | CAUCACAACCUGU | 409 | UGAACAGGUUGUGAU |
| | ACCUGUUCC | | UCA | | GUCAUG |
| 26 | AUGACAUCACAA | 218 | AUCACAACCUGUU | 410 | UGGAACAGGUUGUGA |
| | CCUGUUCCC | | CCA | | UGUCAU |
| 27 | UGACAUCACAAC | 219 | UCACAACCUGUU | 411 | UGGGAACAGGUUGUG |
| | CUGUUCCCA | | CCCA | | AUGUCA |
| 28 | ACAUCACAACCU | 220 | ACAACCUGUUCC | 412 | UUUGGGAACAGGUUG |
| | GUUCCCAAU | | CAAA | | UGAUGU |
| 29 | CAUCACAACCUG | 221 | CAACCUGUUCCC | 413 | UAUUGGGAACAGGUU |
| | UUCCCAAUG | | AAUA | | GUGAUG |
| 30 | UCACAACCUGU | 222 | ACCUGUUCCCAA | 414 | UUCAUUGGGAACAGG |
| | UCCCAAUGAG | | UGAA | | UUGUGA |
| 31 | CACAACCUGUU | 223 | CCUGUUCCCAAU | 415 | UCUCAUUGGGAACAG |
| | CCCAAUGAGA | | GAGA | | GUUGUG |
| 32 | CAACCUGUUCC | 224 | UGUUCCCAAUGA | 416 | UGUCUCAUUGGGAAC |
| | CAAUGAGACC | | GACA | | AGGUUG |
| 33 | CAUCAUAGUGC | 225 | UAGUGCUCCCAU | 417 | UUUGAUGGGAGCACU |
| | UCCCAUCAAA | | CAAA | | AUGAUG |
| 34 | AUCAUAGUGCU | 226 | AGUGCUCCCAUC | 418 | UUUUGAUGGGAGCAC |
| | CCCAUCAAAU | | AAAA | | UAUGAU |
| 35 | UCAUAGUGCUC | 227 | GUGCUCCCAUCA | 419 | UAUUUGAUGGGAGCA |
| | CCAUCAAAUG | | AAUA | | CUAUGA |
| 36 | CAUAGUGCUCC | 228 | UGCUCCCAUCAAA | 420 | UCAUUUGAUGGGAGC |
| | CAUCAAAUGU | | UGA | | ACUAUG |
| 37 | AAUCAAAGUUAU | 229 | AAGUUAUUGGGA | 421 | UUAGUCCCAAUAACU |
| | UGGGACUAU | | CUAA | | UUGAUU |
| 38 | AUCAAAGUUAUU | 230 | AGUUAUUGGGAC | 422 | UAUAGUCCCAAUAAC |
| | GGGACUAUC | | UAUA | | UUUGAU |
| 39 | UCAAAGUUAUU | 231 | GUUAUUGGGACU | 423 | UGAUAGUCCCAAUAA |
| | GGGACUAUCC | | AUCA | | CUUUGA |
| 40 | CAAAGUUAUUG | 232 | UUAUUGGGACUA | 424 | UGGAUAGUCCCAAUA |
| | GGACUAUCCA | | UCCA | | ACUUUG |
| 41 | AAAGUUAUUGG | 233 | UAUUGGGACUAU | 425 | UUGGAUAGUCCCAAU |
| | GACUAUCCAG | | CCAA | | AACUUU |
| 42 | AAGUUAUUGGG | 234 | AUUGGGACUAUC | 426 | UCUGGAUAGUCCCAA |
| | ACUAUCCAGA | | CAGA | | UAACUU |
| 43 | AGUUAUUGGGA | 235 | UUGGGACUAUCC | 427 | UUCUGGAUAGUCCCA |
| | CUAUCCAGAU | | AGAA | | AUAACU |
| 44 | GUUAUUGGGAC | 236 | UGGGACUAUCCA | 428 | UAUCUGGAUAGUCCC |
| | UAUCCAGAUC | | GAUA | | AAUAAC |
| 45 | UUAUUGGGACU | 237 | GGGACUAUCCAG | 429 | UGAUCUGGAUAGUCC |
| | AUCCAGAUCU | | AUCA | | CAAUAA |
| 46 | UAUUGGGACUA | 238 | GGACUAUCCAGA | 430 | UAGAUCUGGAUAGUC |
| | UCCAGAUCUU | | UCUA | | CCAAUA |
| 47 | AUUGGGACUAU | 239 | GACUAUCCAGAU | 431 | UAAGAUCUGGAUAGU |
| | CCAGAUCUUG | | CUUA | | CCCAAU |
| 48 | UUGGGACUAUC | 240 | ACUAUCCAGAUCU | 432 | UCAAGAUCUGGAUAG |
| | CAGAUCUUGU | | UGA | | UCCCAA |
| 49 | UGGGACUAUCC | 241 | CUAUCCAGAUCU | 433 | UACAAGAUCUGGAUA |
| | AGAUCUUGUG | | UGUA | | GUCCCA |
| 50 | GGACUAUCCAG | 242 | AUCCAGAUCUUG | 434 | UACACAAGAUCUGGA |
| | AUCUUGUGUG | | UGUA | | UAGUCC |
| 51 | ACUAUCCAGAUC | 243 | CCAGAUCUUGUG | 435 | UCCACACAAGAUCUG |
| | UUGUGUGGC | | UGGA | | GAUAGU |
| 52 | AUUUUGGCAUC | 244 | GGCAUCUGCUUC | 436 | UAAGGAAGCAGAUGC |
| | UGCUUCCUUC | | CUUA | | CAAAAU |
| 53 | UUUUGGCAUCU | 245 | GCAUCUGCUUCC | 437 | UGAAGGAAGCAGAUG |
| | GCUUCCUUCU | | UUCA | | CCAAAA |
| 54 | UUGGCAUCUGC | 246 | AUCUGCUUCCUU | 438 | UGAGAAGGAAGCAGA |
| | UUCCUUCUCU | | CUCA | | UGCCAA |
| 55 | UGGCAUCUGCU | 247 | UCUGCUUCCUUC | 439 | UAGAGAAGGAAGCAG |
| | UCCUUCUCUC | | UCUA | | AUGCCA |
| 56 | GCAUCUGCUUC | 248 | UGCUUCCUUCUC | 440 | UGGAGAGAAGGAAGC |
| | CUUCUCUCCA | | UCCA | | AGAUGC |
| 57 | CAUCUGCUUCC | 249 | GCUUCCUUCUCU | 441 | UUGGAGAGAAGGAAG |
| | UUCUCUCCAA | | CCAA | | CAGAUG |
| 58 | GCUUCCUUCUC | 250 | CUUCUCUCCAAAU | 442 | UAAAUUUGGAGAGAA |
| | UCCAAAUUUU | | UUA | | GGAAGC |
| 59 | CUUCCUUCUCU | 251 | UUCUCUCCAAAUU | 443 | UAAAAUUUGGAGAGA |
| | CCAAAUUUUA | | UUA | | AGGAAG |
| 60 | UUCCUUCUCUC | 252 | UCUCUCCAAAUUU | 444 | UUAAAAUUUGGAGAG |
| | CAAAUUUUAC | | UAA | | AAGGAA |
| 61 | UCCUUCUCUCC | 253 | CUCUCCAAAUUUU | 445 | UGUAAAAUUUGGAGA |
| | AAAUUUUACC | | ACA | | GAAGGA |
| 62 | CCUUCUCUCCA | 254 | UCUCCAAAUUUUA | 446 | UGGUAAAAUUUGGAG |
| | AAUUUUACCC | | CCA | | AGAAGG |
| 63 | CUUCUCUCCAAA | 255 | CUCCAAAUUUUAC | 447 | UGGGUAAAAUUUGGA |
| | UUUUACCCA | | CCA | | GAGAAG |
| 64 | UUCUCUCCAAAU | 256 | UCCAAAUUUUACC | 448 | UUGGGUAAAAUUUGG |
| | UUUACCCAA | | CAA | | AGAGAA |
| 65 | UCUCUCCAAAUU | 257 | CCAAAUUUUACCC | 449 | UUUGGGUAAAAUUUG |
| | UUACCCAAG | | AAA | | GAGAGA |
| 66 | UCUCCAAAUUUU | 258 | AAAUUUUACCCAA | 450 | UACUUGGGUAAAAUU |
| | ACCCAAGUG | | GUA | | UGGAGA |
| 67 | CUCCAAAUUUUA | 259 | AAUUUUACCCAAG | 451 | UCACUUGGGUAAAAU |
| | CCCAAGUGA | | UGA | | UUGGAG |
| 68 | UCCAAAUUUUAC | 260 | AUUUUACCCAAGU | 452 | UUCACUUGGGUAAAA |
| | CCAAGUGAC | | GAA | | UUUGGA |
| 69 | CAAAUUUUACCC | 261 | UUUACCCAAGUG | 453 | UAGUCACUUGGGUAA |
| | AAGUGACUU | | ACUA | | AAUUUG |
| 70 | AAAUUUUACCCA | 262 | UUACCCAAGUGA | 454 | UAAGUCACUUGGGUA |
| | AGUGACUUC | | CUUA | | AAAUUU |
| 71 | AAUUUUACCCAA | 263 | UACCCAAGUGAC | 455 | UGAAGUCACUUGGGU |
| | GUGACUUCU | | UUCA | | AAAAUU |
| 72 | AUUUUACCCAAG | 264 | ACCCAAGUGACU | 456 | UAGAAGUCACUUGGG |
| | UGACUUCUA | | UCUA | | UAAAAU |
| 73 | UUUUACCCAAG | 265 | CCCAAGUGACUU | 457 | UUAGAAGUCACUUGG |
| | UGACUUCUAC | | CUAA | | GUAAAA |
| 74 | UUUACCCAAGU | 266 | CCAAGUGACUUC | 458 | UGUAGAAGUCACUUG |
| | GACUUCUACA | | UACA | | GGUAAA |
| 75 | UUACCCAAGUG | 267 | CAAGUGACUUCU | 459 | UUGUAGAAGUCACUU |
| | ACUUCUACAC | | ACAA | | GGGUAA |
| 76 | UACCCAAGUGA | 268 | AAGUGACUUCUA | 460 | UGUGUAGAAGUCACU |
| | CUUCUACACU | | CACA | | UGGGUA |
| 77 | ACCCAAGUGAC | 269 | AGUGACUUCUAC | 461 | UAGUGUAGAAGUCAC |
| | UUCUACACUG | | ACUA | | UUGGGU |
| 78 | CCAAGUGACUU | 270 | UGACUUCUACAC | 462 | UACAGUGUAGAAGUC |
| | CUACACUGUU | | UGUA | | ACUUGG |
| 79 | CAAGUGACUUC | 271 | GACUUCUACACU | 463 | UAACAGUGUAGAAGU |
| | UACACUGUUG | | GUUA | | CACUUG |
| 80 | AAGUGACUUCU | 272 | ACUUCUACACUG | 464 | UCAACAGUGUAGAAG |
| | ACACUGUUGA | | UUGA | | UCACUU |
| 81 | UGACUUCUACA | 273 | UCUACACUGUUG | 465 | UGUUCAACAGUGUAG |
| | CUGUUGAACU | | AACA | | AAGUCA |
| 82 | GACUUCUACAC | 274 | CUACACUGUUGA | 466 | UAGUUCAACAGUGUA |
| | UGUUGAACUC | | ACUA | | GAAGUC |
| 83 | CUUCUACACUG | 275 | ACACUGUUGAAC | 467 | UAGAGUUCAACAGUG |
| | UUGAACUCUG | | UCUA | | UAGAAG |
| 84 | UCUACACUGUU | 276 | ACUGUUGAACUC | 468 | UGCAGAGUUCAACAG |
| | GAACUCUGCU | | UGCA | | UGUAGA |
| 85 | CUACACUGUUG | 277 | CUGUUGAACUCU | 469 | UAGCAGAGUUCAACA |
| | AACUCUGCUU | | GCUA | | GUGUAG |
| 86 | UACACUGUUGA | 278 | UGUUGAACUCUG | 470 | UAAGCAGAGUUCAAC |
| | ACUCUGCUUA | | CUUA | | AGUGUA |
| 87 | CACUGUUGAAC | 279 | UUGAACUCUGCU | 471 | UGUAAGCAGAGUUCA |
| | UCUGCUUACC | | UACA | | ACAGUG |
| 88 | ACUGUUGAACU | 280 | UGAACUCUGCUU | 472 | UGGUAAGCAGAGUUC |
| | CUGCUUACCC | | ACCA | | AACAGU |
| 89 | CUGUUGAACUC | 281 | GAACUCUGCUUA | 473 | UGGGUAAGCAGAGUU |
| | UGCUUACCCA | | CCCA | | CAACAG |
| 90 | UGUUGAACUCU | 282 | AACUCUGCUUAC | 474 | UUGGGUAAGCAGAGU |
| | GCUUACCCAU | | CCAA | | UCAACA |
| 91 | GUUGAACUCUG | 283 | ACUCUGCUUACC | 475 | UAUGGGUAAGCAGAG |
| | CUUACCCAUU | | CAUA | | UUCAAC |
| 92 | UGAACUCUGCU | 284 | UCUGCUUACCCA | 476 | UGAAUGGGUAAGCAG |
| | UACCCAUUCA | | UUCA | | AGUUCA |
| 93 | AACUCUGCUUA | 285 | UGCUUACCCAUU | 477 | UAUGAAUGGGUAAGC |
| | CCCAUUCAUA | | CAUA | | AGAGUU |
| 94 | ACUCUGCUUAC | 286 | GCUUACCCAUUC | 478 | UUAUGAAUGGGUAAG |
| | CCAUUCAUAG | | AUAA | | CAGAGU |
| 95 | CUCUGCUUACC | 287 | CUUACCCAUUCAU | 479 | UCUAUGAAUGGGUAA |
| | CAUUCAUAGG | | AGA | | GCAGAG |
| 96 | UCUGCUUACCC | 288 | UUACCCAUUCAUA | 480 | UCCUAUGAAUGGGUA |
| | AUUCAUAGGA | | GGA | | AGCAGA |
| 97 | UGCUUACCCAU | 289 | ACCCAUUCAUAG | 481 | UGUCCUAUGAAUGGG |
| | UCAUAGGACC | | GACA | | UAAGCA |
| 98 | GCUUACCCAUU | 290 | CCCAUUCAUAGG | 482 | UGGUCCUAUGAAUGG |
| | CAUAGGACCC | | ACCA | | GUAAGC |
| 99 | CUUACCCAUUCA | 291 | CCAUUCAUAGGA | 483 | UGGGUCCUAUGAAUG |
| | UAGGACCCU | | CCCA | | GGUAAG |
| 100 | UUACCCAUUCAU | 292 | CAUUCAUAGGAC | 484 | UAGGGUCCUAUGAAU |
| | AGGACCCUU | | CCUA | | GGGUAA |
| 101 | UACCCAUUCAUA | 293 | AUUCAUAGGACC | 485 | UAAGGGUCCUAUGAA |
| | GGACCCUUU | | CUUA | | UGGGUA |
| 102 | ACCCAUUCAUAG | 294 | UUCAUAGGACCC | 486 | UAAAGGGUCCUAUGA |
| | GACCCUUUU | | UUUA | | AUGGGU |
| 103 | CCCAUUCAUAG | 295 | UCAUAGGACCCU | 487 | UAAAAGGGUCCUAUG |
| | GACCCUUUUU | | UUUA | | AAUGGG |
| 104 | CCAUUCAUAGG | 296 | CAUAGGACCCUU | 488 | UAAAAAGGGUCCUAU |
| | ACCCUUUUUU | | UUUA | | GAAUGG |
| 105 | CAUAGGACCCU | 297 | GACCCUUUUUUU | 489 | UUAAAAAAAAAGGGU |
| | UUUUUUUUAU | | UUAA | | CCUAUG |
| 106 | UAGGACCCUUU | 298 | CCCUUUUUUUUU | 490 | UGAUAAAAAAAAAGG |
| | UUUUUUAUCA | | AUCA | | GUCCUA |
| 107 | CAUCUCUGGCU | 299 | CUGGCUCUCUAU | 491 | UUUGAUAGAGAGCCA |
| | CUCUAUCAAU | | CAAA | | GAGAUG |
| 108 | AUCUCUGGCUC | 300 | UGGCUCUCUAUC | 492 | UAUUGAUAGAGAGCC |
| | UCUAUCAAUC | | AAUA | | AGAGAU |
| 109 | GUUAACCAAGC | 301 | CCAAGCUUUUGG | 493 | UGCACCAAAAGCUUG |
| | UUUUGGUGCA | | UGCA | | GUUAAC |
| 110 | UAACCAAGCUUU | 302 | AAGCUUUUGGUG | 494 | UAUGCACCAAAAGCU |
| | UGGUGCAUA | | CAUA | | UGGUUA |
| 111 | AACCAAGCUUUU | 303 | AGCUUUUGGUGC | 495 | UUAUGCACCAAAAGC |
| | GGUGCAUAG | | AUAA | | UUGGUU |
| 112 | ACCAAGCUUUU | 304 | GCUUUUGGUGCA | 496 | UCUAUGCACCAAAAG |
| | GGUGCAUAGC | | UAGA | | CUUGGU |
| 113 | CCAAGCUUUUG | 305 | CUUUUGGUGCAU | 497 | UGCUAUGCACCAAAA |
| | GUGCAUAGCA | | AGCA | | GCUUGG |
| 114 | CAAGCUUUUGG | 306 | UUUUGGUGCAUA | 498 | UUGCUAUGCACCAAA |
| | UGCAUAGCAG | | GCAA | | AGCUUG |
| 115 | AAGCUUUUGGU | 307 | UUUGGUGCAUAG | 499 | UCUGCUAUGCACCAA |
| | GCAUAGCAGC | | CAGA | | AAGCUU |
| 116 | AGCUUUUGGUG | 308 | UUGGUGCAUAGC | 500 | UGCUGCUAUGCACCA |
| | CAUAGCAGCC | | AGCA | | AAAGCU |
| 117 | GCUUUUGGUGC | 309 | UGGUGCAUAGCA | 501 | UGGCUGCUAUGCACC |
| | AUAGCAGCCU | | GCCA | | AAAAGC |
| 118 | UUGGUGCAUAG | 310 | GCAUAGCAGCCU | 502 | UACCAGGCUGCUAUG |
| | CAGCCUGGUU | | GGUA | | CACCAA |
| 119 | UGGUGCAUAGC | 311 | CAUAGCAGCCUG | 503 | UAACCAGGCUGCUAU |
| | AGCCUGGUUG | | GUUA | | GCACCA |
| 120 | AGCAGCCUGGU | 312 | CCUGGUUGGAAG | 504 | UAUGCUUCCAACCAG |
| | UGGAAGCAUU | | CAUA | | GCUGCU |
| 121 | GCAGCCUGGUU | 313 | CUGGUUGGAAGC | 505 | UAAUGCUUCCAACCA |
| | GGAAGCAUUC | | AUUA | | GGCUGC |
| 122 | CAGCCUGGUUG | 314 | UGGUUGGAAGCA | 506 | UGAAUGCUUCCAACC |
| | GAAGCAUUCU | | UUCA | | AGGCUG |
| 123 | AGCCUGGUUGG | 315 | GGUUGGAAGCAU | 507 | UAGAAUGCUUCCAAC |
| | AAGCAUUCUG | | UCUA | | CAGGCU |
| 124 | GCCUGGUUGGA | 316 | GUUGGAAGCAUU | 508 | UCAGAAUGCUUCCAA |
| | AGCAUUCUGA | | CUGA | | CCAGGC |
| 125 | CUGGUUGGAAG | 317 | UGGAAGCAUUCU | 509 | UCUCAGAAUGCUUCC |
| | CAUUCUGAGU | | GAGA | | AACCAG |
| 126 | GCCCUGGUGGG | 318 | GGUGGGUUUCAU | 510 | UAUAAUGAAACCCAC |
| | UUUCAUUAUC | | UAUA | | CAGGGC |
| 127 | CCCUGGUGGGU | 319 | GUGGGUUUCAUU | 511 | UGAUAAUGAAACCCA |
| | UUCAUUAUCC | | AUCA | | CCAGGG |
| 128 | CCUGGUGGGUU | 320 | UGGGUUUCAUUA | 512 | UGGAUAAUGAAACCC |
| | UCAUUAUCCU | | UCCA | | ACCAGG |
| 129 | CUGGUGGGUUU | 321 | GGGUUUCAUUAU | 513 | UAGGAUAAUGAAACC |
| | CAUUAUCCUG | | CCUA | | CACCAG |
| 130 | UGGUGGGUUUC | 322 | GGUUUCAUUAUC | 514 | UCAGGAUAAUGAAAC |
| | AUUAUCCUGU | | CUGA | | CCACCA |
| 131 | GGGUUUCAUUA | 323 | UCAUUAUCCUGU | 515 | UCAGACAGGAUAAUG |
| | UCCUGUCUGU | | CUGA | | AAACCC |
| 132 | GUUUCAUUAUC | 324 | AUUAUCCUGUCU | 516 | UGACAGACAGGAUAA |
| | CUGUCUGUCA | | GUCA | | UGAAAC |
| 133 | CUGCAGUGUGA | 325 | GUGUGAGUUGGA | 517 | UUUGUCCAACUCACA |
| | GUUGGACAAA | | CAAA | | CUGCAG |
| 134 | UGCAGUGUGAG | 326 | UGUGAGUUGGAC | 518 | UUUUGUCCAACUCAC |
| | UUGGACAAAA | | AAAA | | ACUGCA |
| 135 | GCAGUGUGAGU | 327 | GUGAGUUGGACA | 519 | UUUUUGUCCAACUCA |
| | UGGACAAAAA | | AAAA | | CACUGC |
| 136 | CAGUGUGAGUU | 328 | UGAGUUGGACAA | 520 | UUUUUUGUCCAACUC |
| | GGACAAAAAU | | AAAA | | ACACUG |
| 137 | AGUGUGAGUUG | 329 | GAGUUGGACAAA | 521 | UAUUUUUGUCCAACU |
| | GACAAAAAUA | | AAUA | | CACACU |
| 138 | GUGUGAGUUGG | 330 | AGUUGGACAAAAA | 522 | UUAUUUUUGUCCAAC |
| | ACAAAAAUAA | | UAA | | UCACAC |
| 139 | UGUGAGUUGGA | 331 | GUUGGACAAAAAU | 523 | UUUAUUUUUGUCCAA |
| | CAAAAAUAAU | | AAA | | CUCACA |
| 140 | GUGAGUUGGAC | 332 | UUGGACAAAAAUA | 524 | UAUUAUUUUUGUCCA |
| | AAAAAUAAUA | | AUA | | ACUCAC |
| 141 | UACCACGGACU | 333 | CGGACUGCUAUA | 525 | UCUGUAUAGCAGUCC |
| | GCUAUACAGC | | CAGA | | GUGGUA |
| 142 | UCUGAUGCUGA | 334 | UGCUGAUUUGCA | 526 | UGAGUGCAAAUCAGC |
| | UUUGCACUCU | | CUCA | | AUCAGA |
| 143 | CUGAUGCUGAU | 335 | GCUGAUUUGCAC | 527 | UAGAGUGCAAAUCAG |
| | UUGCACUCUG | | UCUA | | CAUCAG |
| 144 | CUGAUUUGCAC | 336 | UUGCACUCUGCU | 528 | UUCCAGCAGAGUGCA |
| | UCUGCUGGAA | | GGAA | | AAUCAG |
| 145 | UGAUUUGCACU | 337 | UGCACUCUGCUG | 529 | UUUCCAGCAGAGUGC |
| | CUGCUGGAAU | | GAAA | | AAAUCA |
| 146 | UUUGCACUCUG | 338 | ACUCUGCUGGAA | 530 | UGAAUUCCAGCAGAG |
| | CUGGAAUUCU | | UUCA | | UGCAAA |
| 147 | UUGCACUCUGC | 339 | CUCUGCUGGAAU | 531 | UAGAAUUCCAGCAGA |
| | UGGAAUUCUG | | UCUA | | GUGCAA |
| 148 | CUCUGCUGGAA | 340 | CUGGAAUUCUGC | 532 | UUAGGCAGAAUUCCA |
| | UUCUGCCUAG | | CUAA | | GCAGAG |
| 149 | UGCUGGAAUUC | 341 | GAAUUCUGCCUA | 533 | UAGCUAGGCAGAAUU |
| | UGCCUAGCUG | | GCUA | | CCAGCA |
| 150 | GCUGGAAUUCU | 342 | AAUUCUGCCUAG | 534 | UCAGCUAGGCAGAAU |
| | GCCUAGCUGU | | CUGA | | UCCAGC |
| 151 | CUGGAAUUCUG | 343 | AUUCUGCCUAGC | 535 | UACAGCUAGGCAGAA |
| | CCUAGCUGUG | | UGUA | | UUCCAG |
| 152 | UGGAAUUCUGC | 344 | UUCUGCCUAGCU | 536 | UCACAGCUAGGCAGA |
| | CUAGCUGUGC | | GUGA | | AUUCCA |
| 153 | CUGCGGUGGAA | 345 | GUGGAAACAGGC | 537 | UUAAGCCUGUUUCCA |
| | ACAGGCUUAC | | UUAA | | CCGCAG |
| 154 | UGCGGUGGAAA | 346 | UGGAAACAGGCU | 538 | UGUAAGCCUGUUUCC |
| | CAGGCUUACU | | UACA | | ACCGCA |
| 155 | GCGGUGGAAAC | 347 | GGAAACAGGCUU | 539 | UAGUAAGCCUGUUUC |
| | AGGCUUACUC | | ACUA | | CACCGC |
| 156 | CGGUGGAAACA | 348 | GAAACAGGCUUA | 540 | UGAGUAAGCCUGUUU |
| | GGCUUACUCU | | CUCA | | CCACCG |
| 157 | UGGAAACAGGC | 349 | ACAGGCUUACUC | 54″ | UUCAGAGUAAGCCUG |
| | UUACUCUGAC | | UGAA | | UUUCCA |
| 158 | GGAAACAGGCU | 350 | CAGGCUUACUCU | 542 | UGUCAGAGUAAGCCU |
| | UACUCUGACU | | GACA | | GUUUCC |
| 159 | GAAACAGGCUU | 351 | AGGCUUACUCUG | 543 | UAGUCAGAGUAAGCC |
| | ACUCUGACUU | | ACUA | | UGUUUC |
| 160 | AAACAGGCUUAC | 352 | GGCUUACUCUGA | 544 | UAAGUCAGAGUAAGC |
| | UCUGACUUC | | CUUA | | CUGUUU |
| 161 | AACAGGCUUAC | 353 | GCUUACUCUGAC | 545 | UGAAGUCAGAGUAAG |
| | UCUGACUUCC | | UUCA | | CCUGUU |
| 162 | ACAGGCUUACU | 354 | CUUACUCUGACU | 546 | UGGAAGUCAGAGUAA |
| | CUGACUUCCC | | UCCA | | GCCUGU |
| 163 | CAGGCUUACUC | 355 | UUACUCUGACUU | 547 | UGGGAAGUCAGAGUA |
| | UGACUUCCCU | | CCCA | | AGCCUG |
| 164 | AGGCUUACUCU | 356 | UACUCUGACUUC | 548 | UAGGGAAGUCAGAGU |
| | GACUUCCCUG | | CCUA | | AAGCCU |
| 165 | GGCUUACUCUG | 357 | ACUCUGACUUCC | 549 | UCAGGGAAGUCAGAG |
| | ACUUCCCUGG | | CUGA | | UAAGCC |
| 166 | UGUACUUUUCC | 358 | UUUUCCUGCCUC | 550 | UUGUGAGGCAGGAAA |
| | UGCCUCACAG | | ACAA | | AGUACA |
| 167 | CUUUUCCUGCC | 359 | CCUGCCUCACAG | 551 | UUAACUGUGAGGCAG |
| | UCACAGUUAC | | UUAA | | GAAAAG |
| 168 | UUUUCCUGCCU | 360 | CUGCCUCACAGU | 552 | UGUAACUGUGAGGCA |
| | CACAGUUACA | | UACA | | GGAAAA |
| 169 | UUUCCUGCCUC | 361 | UGCCUCACAGUU | 553 | UUGUAACUGUGAGGC |
| | ACAGUUACAU | | ACAA | | AGGAAA |
| 170 | GAAGAACUAUU | 362 | ACUAUUGACUUC | 554 | UUAAGAAGUCAAUAG |
| | GACUUCUUAA | | UUAA | | UUCUUC |
| 171 | AGAACUAUUGAC | 363 | UAUUGACUUCUU | 555 | UCUUAAGAAGUCAAU |
| | UUCUUAAGA | | AAGA | | AGUUCU |
| 172 | GAACUAUUGAC | 364 | AUUGACUUCUUAA | 556 | UUCUUAAGAAGUCAA |
| | UUCUUAAGAA | | GAA | | UAGUUC |
| 173 | UGACUUCUUAA | 365 | UCUUAAGAAAAAA | 557 | UCCUUUUUUCUUAAG |
| | GAAAAAAGGG | | GGA | | AAGUCA |
| 174 | GACUUCUUAAG | 366 | CUUAAGAAAAAAG | 558 | UCCCUUUUUUCUUAA |
| | AAAAAAGGGA | | GGA | | GAAGUC |
| 175 | ACUUCUUAAGAA | 367 | UUAAGAAAAAAGG | 559 | UUCCCUUUUUUCUUA |
| | AAAAGGGAG | | GAA | | AGAAGU |
| 176 | CUUCUUAAGAAA | 368 | UAAGAAAAAAGGG | 560 | UCUCCCUUUUUUCUU |
| | AAAGGGAGA | | AGA | | AAGAAG |
| 177 | UUCUUAAGAAAA | 369 | AAGAAAAAAGGGA | 561 | UUCUCCCUUUUUUCU |
| | AAGGGAGAA | | GAA | | UAAGAA |
| 178 | CUUAAGAAAAAA | 370 | GAAAAAAGGGAGA | 562 | UUUUCUCCCUUUUUU |
| | GGGAGAAAU | | AAA | | CUUAAG |
| 179 | AAGGGAGAAAUA | 371 | AGAAAUAUUAAUC | 563 | UCUGAUUAAUAUUUC |
| | UUAAUCAGA | | AGA | | UCCCUU |
| 180 | AGGGAGAAAUA | 372 | GAAAUAUUAAUCA | 564 | UUCUGAUUAAUAUUU |
| | UUAAUCAGAA | | GAA | | CUCCCU |
| 181 | GGGAGAAAUAU | 373 | AAAUAUUAAUCAG | 565 | UUUCUGAUUAAUAUU |
| | UAAUCAGAAA | | AAA | | UCUCCC |
| 182 | UCAGAAAGUUG | 374 | AAGUUGAUUCUU | 566 | UCAUAAGAAUCAACU |
| | AUUCUUAUGA | | AUGA | | UUCUGA |
| 183 | CAGAAAGUUGA | 375 | AGUUGAUUCUUA | 567 | UUCAUAAGAAUCAAC |
| | UUCUUAUGAU | | UGAA | | UUUCUG |
| 184 | AUAGAAAAGCAA | 376 | AAAGCAAAGCUUG | 568 | UCUCAAGCUUUGCUU |
| | AGCUUGAGU | | AGA | | UUCUAU |
| 185 | UAGAAAAGCAAA | 377 | AAGCAAAGCUUGA | 569 | UACUCAAGCUUUGCU |
| | GCUUGAGUU | | GUA | | UUUCUA |
| 186 | AGAAAAGCAAAG | 378 | AGCAAAGCUUGA | 570 | UAACUCAAGCUUUGC |
| | CUUGAGUUU | | GUUA | | UUUUCU |
| 187 | GAAAAGCAAAGC | 379 | GCAAAGCUUGAG | 571 | UAAACUCAAGCUUUG |
| | UUGAGUUUC | | UUUA | | CUUUUC |
| 188 | AAAAGCAAAGCU | 380 | CAAAGCUUGAGU | 572 | UGAAACUCAAGCUUU |
| | UGAGUUUCC | | UUCA | | GCUUUU |
| 189 | AAAGCAAAGCUU | 381 | AAAGCUUGAGUU | 573 | UGGAAACUCAAGCUU |
| | GAGUUUCCU | | UCCA | | UGCUUU |
| 190 | AGCAAAGCUUG | 382 | AGCUUGAGUUUC | 574 | UUAGGAAACUCAAGC |
| | AGUUUCCUAA | | CUAA | | UUUGCU |
| 191 | GCAAAGCUUGA | 383 | GCUUGAGUUUCC | 575 | UUUAGGAAACUCAAG |
| | GUUUCCUAAA | | UAAA | | CUUUGC |
| 192 | CAAAGCUUGAG | 384 | CUUGAGUUUCCU | 576 | UUUUAGGAAACUCAA |
| | UUUCCUAAAU | | AAAA | | GCUUUG |

siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100%70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 564,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

A-B-(A′)ⱼ-C-P²-D-P¹—(C′-P¹)ₖ-C′ Formula I;
wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

A-B-(A′)ⱼ-C-P²-D-P¹—(C-P¹)ₖ-C′ Formula II;
wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

E-(A′)ₘ-F Formula III;
wherein E is represented by the formula (C-P¹)₂; F is represented by the formula (C-P²)₃-D-P¹-C-P¹—C, (C-P²)₃-D-P²-C-P²—C, (C-P²)₃-D-P¹-C-P¹-D, or (C-P²)₃-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

A-(A′)ⱼ-C-P²—B—(C-P¹)ₖ-C′ Formula IV;
wherein A is represented by the formula C-P¹-D-P¹; each A′ is represented by the formula C-P²-D-P²; B is represented by the formula D-P¹-C-P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

E-(A′)ₘ-C-P²—F Formula V;
wherein E is represented by the formula (C-P¹)₂; F is represented by the formula D-P¹-C-P¹—C, D-P²-C-P²—C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

A-Bⱼ-E-Bₖ-E-F-Gₗ-D-P¹—C′ Formula VI;
wherein A is represented by the formula C-P¹-D-P¹; each B is represented by the formula C-P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²-C-P²; F is represented by the formula D-P¹-C-P¹; each G is represented by the formula C-P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, l is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

H-Bₘ-Iₙ-A′-Bₒ-H-C Formula VII;
wherein A′ is represented by the formula C-P²-D-P²; each H is represented by the formula (C-P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

Z-((A-P-)ₙ(B-P-)ₘ)_{q}; Formula VIII
wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
   Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

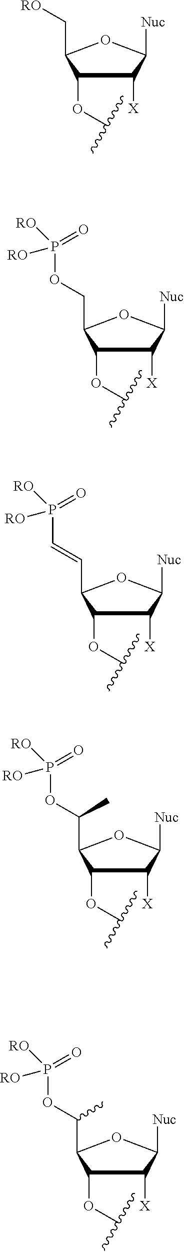

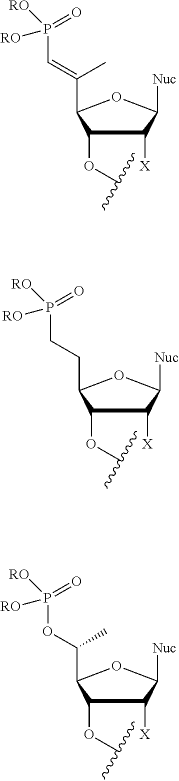

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substituted phosphates as in Formula XII, XIII, and XVI, methylenephosphonates as in Formula XV, or vinyl 5′-vinylphosphonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA—L—RNA | 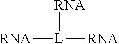 | 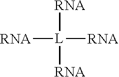 |
| Formula XVII | Formula XX | Formula XXIV |
| 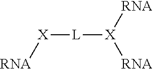 | 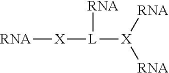 | 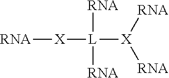 |
| Formula XVIII | Formula XXI | Formula XXV |
| 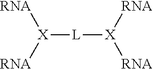 | 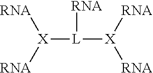 | 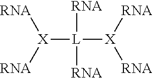 |
| Formula XIX | Formula XXII | Formula XXVI |
| | 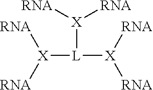 | 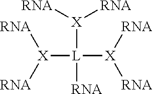 |
| | Formula XXIII | Formula XXVII |
| | | 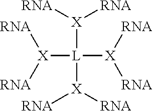 |
| | | Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

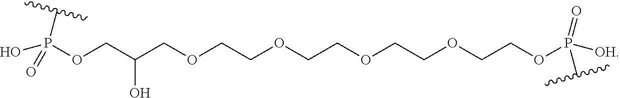 [SMILES: *P(=O)(O)OCCOCCOCCOCCOCC(O)COP(*)(=O)O]

In some embodiments, the linker has a structure of Formula L2:

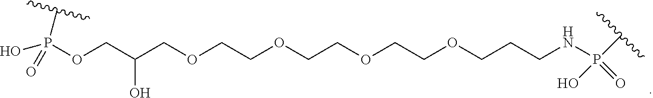 [SMILES: *P(=O)(O)NCCCOCCOCCOCCOCC(O)COP(*)(=O)O]

In some embodiments, the linker has a structure of Formula L3:

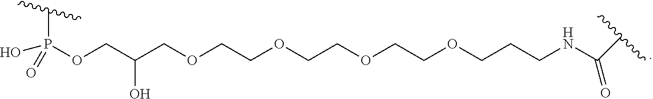 [SMILES: *C(=O)NCCCOCCOCCOCCOCC(O)COP(*)(=O)O]

In some embodiments, the linker has a structure of Formula L4:

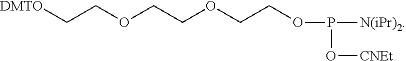

In some embodiments, the linker has a structure of Formula L5:

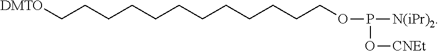

In some embodiments, the linker has a structure of Formula L6:

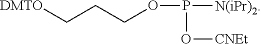

In some embodiments, the linker has a structure of Formula L7, as is shown below:

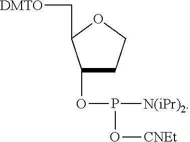

In some embodiments, the linker has a structure of Formula L8:

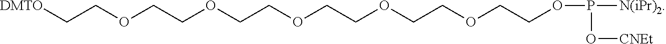

In some embodiments, the linker has a structure of Formula L9:

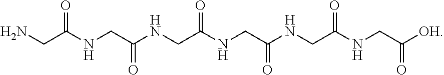 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The MS4A6A-targeting siRNA molecules of the disclosure may be delivered to a subject, thereby treating a neuroinflammatory disease, such as Alzheimer's disease, and/or mitigating phenotypes associated with the neuroinflammatory disease. For example, the siRNA molecules may be delivered to a subject, thereby treating Alzheimer's disease and/or mitigating Alzheimer's disease associated phenotypes (e.g., cognitive impairment or memory loss). Furthermore, the siRNA molecules of the disclosure may also be delivered to a subject having a variant of the MS4A6A gene for which siRNA-mediated gene silencing of the MS4A6A variant gene reduces the expression level of MS4A6A transcript, thereby treating a neuroinflammatory disease (e.g., Alzheimer's disease) or other MS4A-related diseases or disorders.

The disclosure provides methods of treating a subject by way of MS4A6A gene silencing with one or more of the siRNA molecules described herein. The gene silencing may be performed in a subject to silence wild type MS4A6A transcripts, mutant MS4A6A transcripts, splice isoforms of MS4A6A transcripts, and/or overexpressed MS4A6A transcripts thereof, relative to a healthy subject. The method may include delivering to the CNS or affected tissues of the subject (e.g., a human) the siRNA molecules of the disclosure or a pharmaceutical composition containing the same by any appropriate route of administration (e.g., intracerebroventricular, intrathecal injection, intrastriatal injection, intra-cisterna magna injection by catheterization, intraparenchymal injection, intravenous injection, subcutaneous injection, or intramuscular injection). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, a neuroinflammatory disease (e.g., Alzheimer's disease) and/or any other medical risk(s) associated with gain of function mutations in the MS4A6A gene. Subjects that may be treated with the siRNA molecules disclosed herein may include, for example, humans, monkeys, rats, mice, pigs, and other mammals containing at least one orthologous copy of the MS4A6A gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues of the subject (e.g., by way of intracerebroventricular, intrastriatally, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. MS4A6A Knockdown

### Objective

This Example describes the results of a series of experiments undertaken to investigate the ability of siRNA molecules complementary to specific regions within a human MS4A6A mRNA transcript to effectuate reduced expression of the MS4A6A gene.

### Materials and Methods

HEK cells stably expressing MS4A6A (HEK-MS4A6A) were actively transfected with MS4A siRNA at either 20 nM or 0.2 nM concentration. After 24 hours, cells were lysed, and mRNA levels of MS4A6A and a housekeeping gene (ATP5B) were assessed via reverse transcription-quantitative polymerase chain reaction (RT-qPCR), using standard reagents and Applied Biosystems TaqMan Assays. Results are presented as the percent residual MS4A6A mRNA relative to untreated control cells in the same assay.

### Results

Cells were treated with an siRNA molecule of the disclosure having an antisense strand and a sense strand as shown in Tables 3 and 4, below. The knockdown efficiency of the siRNA molecule was measured as the residual mRNA expression as a percent of untreated at 20 nM and 0.2 nM. The knockdown results are reported in Table 3 (20 nM) and Table 4 (0.2 nM).

TABLE 3
MS4A6A knockdown with siRNA molecules
of the disclosure at 20 nM

| Sense | Antisense | Residual mRNA expression |
|---|---|---|
| SEQ ID NO: | SEQ ID NO: | (% of untreated at 20 nM) |
| 193 | 385 | 18.03 |
| 194 | 386 | 18.80 |
| 195 | 387 | 36.39 |
| 196 | 388 | 38.52 |
| 197 | 389 | 26.72 |
| 198 | 390 | 19.26 |
| 199 | 391 | 30.97 |
| 200 | 392 | 13.13 |
| 201 | 393 | 35.24 |
| 202 | 394 | 61.09 |
| 203 | 395 | 13.02 |
| 204 | 396 | 40.99 |
| 205 | 397 | 38.33 |
| 206 | 398 | 23.69 |
| 207 | 399 | 25.35 |
| 208 | 400 | 17.69 |
| 209 | 401 | 23.15 |
| 210 | 402 | 66.35 |
| 211 | 403 | 29.93 |
| 212 | 404 | 69.37 |
| 213 | 405 | 16.93 |
| 214 | 406 | 44.02 |
| 215 | 407 | 58.46 |
| 216 | 408 | 34.53 |
| 217 | 409 | 47.31 |
| 218 | 410 | 36.04 |
| 219 | 411 | 43.77 |
| 220 | 412 | 13.84 |
| 221 | 413 | 21.23 |
| 222 | 414 | 40.14 |
| 223 | 415 | 30.23 |
| 224 | 416 | 24.93 |
| 225 | 417 | 28.60 |
| 226 | 418 | 29.28 |
| 227 | 419 | 69.21 |
| 228 | 420 | 88.56 |
| 229 | 421 | 37.11 |
| 230 | 422 | 32.29 |
| 231 | 423 | 75.50 |
| 232 | 424 | 59.48 |
| 233 | 425 | 43.15 |
| 234 | 426 | 22.27 |
| 235 | 427 | 69.13 |
| 236 | 428 | 73.87 |
| 237 | 429 | 46.62 |
| 238 | 430 | 33.26 |
| 239 | 431 | 93.42 |
| 240 | 432 | 35.95 |
| 241 | 433 | 16.50 |
| 242 | 434 | 32.21 |
| 243 | 435 | 14.18 |
| 244 | 436 | 57.74 |
| 245 | 437 | 16.51 |
| 246 | 438 | 14.35 |
| 247 | 439 | 11.01 |
| 248 | 440 | 46.29 |
| 249 | 441 | 19.20 |
| 250 | 442 | 23.30 |
| 251 | 443 | 26.75 |
| 252 | 444 | 25.87 |
| 253 | 445 | 19.36 |
| 254 | 446 | 34.32 |
| 255 | 447 | 48.69 |
| 256 | 448 | 20.73 |
| 257 | 449 | 19.54 |
| 258 | 450 | 24.21 |
| 259 | 451 | 38.72 |
| 260 | 452 | 19.56 |
| 261 | 453 | 54.93 |
| 262 | 454 | 36.84 |
| 263 | 455 | 90.59 |
| 264 | 456 | 59.04 |
| 265 | 457 | 36.31 |
| 266 | 458 | 16.14 |
| 267 | 459 | 33.63 |
| 268 | 460 | 31.75 |
| 269 | 461 | 27.08 |
| 270 | 462 | 35.96 |
| 271 | 463 | 39.24 |
| 272 | 464 | 13.78 |
| 273 | 465 | 22.56 |
| 274 | 466 | 27.36 |
| 275 | 467 | 19.37 |
| 276 | 468 | 28.93 |
| 277 | 469 | 22.03 |
| 278 | 470 | 17.25 |
| 279 | 471 | 27.92 |
| 280 | 472 | 28.01 |
| 281 | 473 | 23.14 |
| 282 | 474 | 27.67 |
| 283 | 475 | 21.80 |
| 284 | 476 | 18.17 |
| 285 | 477 | 72.05 |
| 286 | 478 | 33.01 |
| 287 | 479 | 26.51 |
| 288 | 480 | 48.45 |
| 289 | 481 | 41.62 |
| 290 | 482 | 37.47 |
| 291 | 483 | 33.03 |
| 292 | 484 | 23.88 |
| 293 | 485 | 20.44 |
| 294 | 486 | 61.06 |
| 295 | 487 | 51.88 |
| 296 | 488 | 41.71 |
| 297 | 489 | 54.85 |
| 298 | 490 | 19.34 |
| 299 | 491 | 29.00 |
| 300 | 492 | 19.94 |
| 301 | 493 | 11.57 |
| 302 | 494 | 16.76 |
| 303 | 495 | 22.63 |
| 304 | 496 | 16.23 |
| 305 | 497 | 24.11 |
| 306 | 498 | 13.22 |
| 307 | 499 | 17.45 |
| 308 | 500 | 99.81 |
| 309 | 501 | 48.18 |
| 310 | 502 | 30.25 |
| 311 | 503 | 24.28 |
| 312 | 504 | 13.45 |
| 313 | 505 | 77.97 |
| 314 | 506 | 27.48 |
| 315 | 507 | 15.53 |
| 316 | 508 | 10.30 |
| 317 | 509 | 22.49 |
| 318 | 510 | 12.22 |
| 319 | 511 | 15.33 |
| 320 | 512 | 28.58 |
| 321 | 513 | 17.57 |
| 322 | 514 | 16.29 |
| 323 | 515 | 12.16 |
| 324 | 516 | 23.40 |
| 325 | 517 | 32.36 |
| 326 | 518 | 21.39 |
| 327 | 519 | 32.31 |
| 328 | 520 | 33.87 |
| 329 | 521 | 50.34 |
| 330 | 522 | 28.95 |
| 331 | 523 | 15.44 |
| 332 | 524 | 26.94 |
| 333 | 525 | 28.93 |
| 334 | 526 | 15.00 |
| 335 | 527 | 13.24 |
| 336 | 528 | 49.70 |
| 337 | 529 | 63.30 |
| 338 | 530 | 53.25 |
| 339 | 531 | 14.47 |
| 340 | 532 | 17.91 |
| 341 | 533 | 16.55 |
| 342 | 534 | 16.44 |
| 343 | 535 | 52.40 |
| 344 | 536 | 38.34 |
| 345 | 537 | 29.62 |
| 346 | 538 | 26.80 |
| 347 | 539 | 24.84 |
| 348 | 540 | 20.08 |
| 349 | 541 | 10.93 |
| 350 | 542 | 24.27 |
| 351 | 543 | 21.05 |
| 352 | 544 | 21.08 |
| 353 | 545 | 16.99 |
| 354 | 546 | 28.28 |
| 355 | 547 | 34.30 |
| 356 | 548 | 13.28 |
| 357 | 549 | 36.39 |
| 358 | 550 | 12.89 |
| 359 | 551 | 17.64 |
| 360 | 552 | 34.04 |
| 361 | 553 | 85.84 |
| 362 | 554 | 19.91 |
| 363 | 555 | 15.55 |
| 364 | 556 | 14.83 |
| 365 | 557 | 17.16 |
| 366 | 558 | 17.83 |
| 367 | 559 | 19.61 |
| 368 | 560 | 18.43 |
| 369 | 561 | 34.44 |
| 370 | 562 | 16.32 |
| 371 | 563 | 13.55 |
| 372 | 564 | 13.90 |
| 373 | 565 | 12.93 |
| 374 | 566 | 13.20 |
| 375 | 567 | 9.43 |
| 376 | 568 | 18.28 |
| 377 | 569 | 13.02 |
| 378 | 570 | 15.14 |
| 379 | 571 | 12.61 |
| 380 | 572 | 12.76 |
| 381 | 573 | 12.91 |
| 382 | 574 | 17.17 |
| 383 | 575 | 12.08 |
| 384 | 576 | 12.47 |

TABLE 4
MS4A6A knockdown with siRNA molecules
of the disclosure at 0.2 nM

| Sense | Antisense | Residual mRNA expression |
|---|---|---|
| SEQ ID NO: | SEQ ID NO: | (% of untreated at 0.2 nM) |
| 193 | 385 | 77.87 |
| 194 | 386 | 89.78 |
| 195 | 387 | 94.38 |
| 196 | 388 | 96.59 |
| 197 | 389 | 94.74 |
| 198 | 390 | 93.58 |
| 199 | 391 | 91.20 |
| 200 | 392 | 74.18 |
| 201 | 393 | 83.43 |
| 202 | 394 | 94.08 |
| 203 | 395 | 88.71 |
| 204 | 396 | 104.23 |
| 205 | 397 | 104.69 |
| 206 | 398 | 95.38 |
| 207 | 399 | 96.83 |
| 208 | 400 | 82.48 |
| 209 | 401 | 85.18 |
| 210 | 402 | 106.03 |
| 211 | 403 | 102.54 |
| 212 | 404 | 103.04 |
| 213 | 405 | 70.29 |
| 214 | 406 | 110.34 |
| 215 | 407 | 116.37 |
| 216 | 408 | 116.00 |
| 217 | 409 | 104.73 |
| 218 | 410 | 98.82 |
| 219 | 411 | 102.79 |
| 220 | 412 | 68.64 |
| 221 | 413 | 94.26 |
| 222 | 414 | 95.86 |
| 223 | 415 | 94.96 |
| 224 | 416 | 94.69 |
| 225 | 417 | 96.72 |
| 226 | 418 | 95.79 |
| 227 | 419 | 103.80 |
| 228 | 420 | 102.47 |
| 229 | 421 | 92.68 |
| 230 | 422 | 90.53 |
| 231 | 423 | 101.73 |
| 232 | 424 | 101.30 |
| 233 | 425 | 93.27 |
| 234 | 426 | 91.38 |
| 235 | 427 | 103.52 |
| 236 | 428 | 97.97 |
| 237 | 429 | 86.71 |
| 238 | 430 | 92.40 |
| 239 | 431 | 97.78 |
| 240 | 432 | 88.88 |
| 241 | 433 | 95.23 |
| 242 | 434 | 92.36 |
| 243 | 435 | 78.65 |
| 244 | 436 | 99.18 |
| 245 | 437 | 80.85 |
| 246 | 438 | 72.41 |
| 247 | 439 | 44.18 |
| 248 | 440 | 76.42 |
| 249 | 441 | 81.83 |
| 250 | 442 | 77.71 |
| 251 | 443 | 96.80 |
| 252 | 444 | 94.05 |
| 253 | 445 | 54.93 |
| 254 | 446 | 74.16 |
| 255 | 447 | 91.20 |
| 256 | 448 | 71.87 |
| 257 | 449 | 75.82 |
| 258 | 450 | 87.41 |
| 259 | 451 | 81.12 |
| 260 | 452 | 82.61 |
| 261 | 453 | 81.59 |
| 262 | 454 | 79.52 |
| 263 | 455 | 93.59 |
| 264 | 456 | 91.20 |
| 265 | 457 | 89.25 |
| 266 | 458 | 71.33 |
| 267 | 459 | 86.47 |
| 268 | 460 | 83.82 |
| 269 | 461 | 68.05 |
| 270 | 462 | 89.02 |
| 271 | 463 | 88.70 |
| 272 | 464 | 58.95 |
| 273 | 465 | 82.25 |
| 274 | 466 | 81.79 |
| 275 | 467 | 67.37 |
| 276 | 468 | 72.60 |
| 277 | 469 | 72.49 |
| 278 | 470 | 52.73 |
| 279 | 471 | 73.24 |
| 280 | 472 | 63.73 |
| 281 | 473 | 78.52 |
| 282 | 474 | 90.95 |
| 283 | 475 | 85.17 |
| 284 | 476 | 73.22 |
| 285 | 477 | 96.43 |
| 286 | 478 | 82.44 |
| 287 | 479 | 78.94 |
| 288 | 480 | 91.53 |
| 289 | 481 | 95.83 |
| 290 | 482 | 97.49 |
| 291 | 483 | 99.56 |
| 292 | 484 | 93.72 |
| 293 | 485 | 100.54 |
| 294 | 486 | 105.32 |
| 295 | 487 | 102.95 |
| 296 | 488 | 96.31 |
| 297 | 489 | 87.37 |
| 298 | 490 | 66.45 |
| 299 | 491 | 87.84 |
| 300 | 492 | 83.85 |
| 301 | 493 | 60.06 |
| 302 | 494 | 81.62 |
| 303 | 495 | 86.99 |
| 304 | 496 | 71.10 |
| 305 | 497 | 84.93 |
| 306 | 498 | 72.88 |
| 307 | 499 | 81.82 |
| 308 | 500 | 93.12 |
| 309 | 501 | 92.30 |
| 310 | 502 | 91.66 |
| 311 | 503 | 78.43 |
| 312 | 504 | 73.15 |
| 313 | 505 | 86.83 |
| 314 | 506 | 84.15 |
| 315 | 507 | 90.02 |
| 316 | 508 | 89.28 |
| 317 | 509 | 89.98 |
| 318 | 510 | 68.51 |
| 319 | 511 | 73.08 |
| 320 | 512 | 95.40 |
| 321 | 513 | 81.98 |
| 322 | 514 | 69.68 |
| 323 | 515 | 56.95 |
| 324 | 516 | 80.44 |
| 325 | 517 | 94.66 |
| 326 | 518 | 76.36 |
| 327 | 519 | 87.43 |
| 328 | 520 | 97.72 |
| 329 | 521 | 98.01 |
| 330 | 522 | 89.84 |
| 331 | 523 | 65.22 |
| 332 | 524 | 70.08 |
| 333 | 525 | 90.77 |
| 334 | 526 | 86.39 |
| 335 | 527 | 68.68 |
| 336 | 528 | 94.56 |
| 337 | 529 | 95.49 |
| 338 | 530 | 97.13 |
| 339 | 531 | 77.17 |
| 340 | 532 | 80.63 |
| 341 | 533 | 81.83 |
| 342 | 534 | 86.00 |
| 343 | 535 | 93.63 |
| 344 | 536 | 92.90 |
| 345 | 537 | 93.35 |
| 346 | 538 | 94.56 |
| 347 | 539 | 95.98 |
| 348 | 540 | 93.35 |
| 349 | 541 | 71.68 |
| 350 | 542 | 87.35 |
| 351 | 543 | 84.84 |
| 352 | 544 | 94.60 |
| 353 | 545 | 80.24 |
| 354 | 546 | 92.84 |
| 355 | 547 | 96.35 |
| 356 | 548 | 70.05 |
| 357 | 549 | 94.00 |
| 358 | 550 | 73.56 |
| 359 | 551 | 85.00 |
| 360 | 552 | 88.88 |
| 361 | 553 | 89.62 |
| 362 | 554 | 74.62 |
| 363 | 555 | 69.17 |
| 364 | 556 | 79.70 |
| 365 | 557 | 70.70 |
| 366 | 558 | 82.53 |
| 367 | 559 | 81.92 |
| 368 | 560 | 80.63 |
| 369 | 561 | 97.69 |
| 370 | 562 | 85.52 |
| 371 | 563 | 70.87 |
| 372 | 564 | 68.38 |
| 373 | 565 | 66.82 |
| 374 | 566 | 54.84 |
| 375 | 567 | 30.96 |
| 376 | 568 | 75.64 |
| 377 | 569 | 46.07 |
| 378 | 570 | 44.37 |
| 379 | 571 | 39.95 |
| 380 | 572 | 40.68 |
| 381 | 573 | 43.08 |
| 382 | 574 | 59.07 |
| 383 | 575 | 36.39 |
| 384 | 576 | 43.58 |

### Example 2. IC50 Determination of MS4A6A-Targeting siRNA Molecules

### Objective

This Example describes the results of a series of experiments undertaken to determine the IC50 of siRNA molecules complementary to specific regions within a human MS4A6A mRNA.

### Materials and Methods

HEK cells stably expressing MS4A6A (HEK-MS4A6A) were actively transfected with MS4A siRNA at concentrations of 1 fM to 100 nM for 24 hours. Expression of MS4A6A mRNA was assessed at 72 hours using RT-qPCR as described above, and the IC50 of each compound was calculated.

### Results

Cells were treated with an siRNA molecule of the disclosure having an antisense strand and a sense strand as shown in Table 5, below. The IC50 was calculated and the knockdown results are reported in Table 5.

TABLE 5
IC50 of MS4A6A-targeting siRNA Molecules

| Sense | Antisense | IC50 |
|---|---|---|
| SEQ ID NO: | SEQ ID NO: | (pM) |
| 200 | 392 | 194 |
| 220 | 412 | 665 |
| 246 | 438 | 437 |
| 247 | 439 | 281 |
| 253 | 445 | 964 |
| 254 | 446 | 833 |
| 266 | 458 | 157 |
| 272 | 464 | 126 |
| 272 | 464 | 126 |
| 275 | 467 | 137 |
| 276 | 468 | 510 |
| 278 | 470 | 158 |
| 280 | 472 | 136 |
| 298 | 490 | 84.9 |
| 301 | 493 | 585 |
| 306 | 498 | 442 |
| 312 | 504 | 144 |
| 318 | 510 | 66.5 |
| 319 | 511 | 122 |
| 323 | 515 | 20.7 |
| 331 | 523 | 76.6 |
| 332 | 524 | 81.9 |
| 335 | 527 | 145 |
| 349 | 541 | 81.7 |
| 356 | 548 | 122 |
| 358 | 550 | 78.9 |
| 363 | 555 | 133 |
| 371 | 563 | 193 |
| 372 | 564 | 308 |
| 373 | 565 | 338 |
| 374 | 566 | 171 |
| 375 | 567 | 17.2 |
| 377 | 569 | 48.2 |
| 378 | 570 | 92.8 |
| 379 | 571 | 52.3 |
| 380 | 572 | 50.5 |
| 381 | 573 | 42.9 |
| 382 | 574 | 71.6 |
| 383 | 575 | 7.23 |
| 384 | 576 | 69.9 |

### Example 3. Generating MS4A6A-Targeting siRNA Molecules

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the MS4A6A mRNA target sequence, are shown in Table 1, above. It is appreciated that one of skill in the art could anneal the antisense (AS) strand to the corresponding sense (S) strand to yield a ds-siRNA molecule. Alternatively, one of skill in the art could derive a ss-siRNA molecule using antisense strand only.

### Example 4. Optimizing MS4A6A-Targeting siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 5. Preparation and Administrating MS4A6A-Targeting siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject that is tolerated by the siRNA compositions of the disclosure. Non-limiting examples of siRNA injections into the CNS include intrathecally, intracerebroventricularly, intrastriatally, intraparenchymally, or intra-cisterna magna injection by catheterization. Examples of systemic administration include intravenous, intramuscular, and subcutaneous injection. A physician having ordinary skill in the art can readily determine an effective route of administration.

### Example 6. Methods for the Treatment of a Neuroinflammatory Disease Using MS4A6A-Targeting siRNA Molecules

A subject in need of treatment of a neuroinflammatory disease (e.g., Alzheimer's disease), is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of MS4A6A mRNA or suitable biomarker or a reduction of Alzheimers disease-associated phenotypes) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, intrastriatally, intraparenchymally, intravenously, intramuscularly, subcutaneously, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, intrastriatally, intraparenchymally, intravenously, intramuscularly, subcutaneously, or by intra-cisterna magna injection via catheterization) and condition at a rate tolerable to the patient until the subject has reached a maximum tolerated dose, or until symptoms are ameliorated satisfactorily.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand has complementarity sufficient to hybridize to a region within a membrane-spanning 4-domains A6A (MS4A6A) mRNA transcript having the nucleic acid sequence of any one of SEQ ID NOs: 1-192.

2. The siRNA molecule of claim 1, wherein the antisense strand has at least 70% complementarity to a region of 19, 20, 21, or more contiguous nucleobases within the MS4A6A mRNA transcript having the nucleic acid sequence of any one of SEQ ID NOs: 1-192, optionally wherein the antisense strand has at least 70% complementarity to the MS4A6A mRNA transcript having the nucleic acid sequence of any one of SEQ ID NOs: 1-192.

3. The siRNA molecule of claim 2, wherein the antisense strand has at least 75% complementarity to a region of 21 contiguous nucleobases within the MS4A6A mRNA transcript having the nucleic acid sequence of any one of SEQ ID NOs: 1-192, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the MS4A6A mRNA transcript having the nucleic acid sequence of any one of SEQ ID Nos: 1-192.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the MS4A6A RNA transcript having the nucleic acid sequence of any one of SEQ ID NOs: 1-192.

5. The siRNA molecule of claim 4, wherein the antisense strand comprises from 10 to 30 contiguous nucleotides that are fully complementary to a contiguous polynucleotide segment of equal length within the region of the MS4A6A RNA transcript having the nucleic acid sequence of any one of SEQ ID NOs: 1-192.

6. The siRNA molecule of claim 5, wherein the antisense strand comprises from 12 to 30 contiguous nucleotides that are fully complementary to a contiguous polynucleotide segment of equal length within the region of the MS4A6A RNA transcript having the nucleic acid sequence of any one of SEQ ID NOs: 1-192.

7. The siRNA molecule of claim 6, wherein the antisense strand comprises from 15 to 30 contiguous nucleotides that are fully complementary to a contiguous polynucleotide segment of equal length within the region of the MS4A6A RNA transcript having the nucleic acid sequence of any one of SEQ ID NOs: 1-192.

8. The siRNA molecule of claim 7, wherein the antisense strand comprises from 18 to 30 contiguous nucleotides that are fully complementary to a contiguous polynucleotide segment of equal length within the region of the MS4A6A RNA transcript having the nucleic acid sequence of any one of SEQ ID NOs: 1-192.

9. The siRNA molecule of claim 8, wherein the antisense strand comprises from 18 to 25 contiguous nucleotides that are fully complementary to a contiguous polynucleotide segment of equal length within the region of the MS4A6A RNA transcript having the nucleic acid sequence of any one of SEQ ID NOs: 1-192.

10. The siRNA molecule of any one of claim 9, wherein the antisense strand comprises from 18 to 21 contiguous nucleotides that are fully complementary to a contiguous polynucleotide segment of equal length within the region of the MS4A6A RNA transcript having the nucleic acid sequence of any one of SEQ ID NOs: 1-192.

11. The siRNA molecule of claim 10, wherein the antisense strand comprises 21 contiguous nucleotides that are fully complementary to a contiguous polynucleotide segment of equal length within the region of the MS4A6A RNA transcript having the nucleic acid sequence of any one of SEQ ID NOs: 1-192.

12. The siRNA molecule of any one of claims 1-11, wherein the antisense strand comprises 9 or fewer nucleotide mismatches relative to a region of 21 contiguous nucleobases of the MS4A6A RNA transcript having the nucleic acid sequence of any one of SEQ ID NOs: 1-192, optionally wherein the antisense strand comprises 8 or fewer, 7 or fewer, 6 or fewer, 5 or fewer, 4 or fewer, 3 or fewer, 2 or fewer, or only 1 mismatch relative to the region of the MS4A6A RNA transcript having the nucleic acid sequence of any one of SEQ ID NOs: 1-192.

13. The siRNA molecule of any one of claims 1-12, wherein the antisense strand has a nucleic acid sequence that is at least 85% identical to the nucleic acid sequence of any one of SEQ ID NOs: 385-576.

14. The siRNA molecule of claim 13, wherein the antisense strand has a nucleic acid sequence that is at least 90% identical to the nucleic acid sequence of any one of SEQ ID NOs: 385-576.

15. The siRNA molecule of claim 14, wherein the antisense strand has a nucleic acid sequence that is at least 95% identical to the nucleic acid sequence of SEQ ID NOs: 385-576, optionally wherein the antisense strand has a nucleic acid sequence that is at least 96%, 97%, 98%, or 99% identical to the nucleic acid sequence of any one of SEQ ID NOs: 385-576.

16. The siRNA molecule of claim 15, wherein the antisense strand has the nucleic acid sequence of any one of SEQ ID NOs: 385-576.

17. The siRNA molecule of any one of claims 1-16, wherein the sense strand has a nucleic acid sequence that is at least 85% identical to the nucleic acid sequence of any one of SEQ ID NOs: 193-384.

18. The siRNA molecule of claim 17, wherein the sense strand has a nucleic acid sequence that is at least 90% identical to the nucleic acid sequence of any one of SEQ ID NOs: 193-384.

19. The siRNA molecule of claim 18, wherein the sense strand has a nucleic acid sequence that is at least 95% identical to the nucleic acid sequence of SEQ ID NOs: 193-384, optionally wherein the sense strand has a nucleic acid sequence that is at least 96%, 97%, 98%, or 99% identical to the nucleic acid sequence of any one of SEQ ID NOs: 193-384.

20. The siRNA molecule of claim 19, wherein the sense strand has the nucleic acid sequence of any one of SEQ ID NOs: 193-384.

21. The siRNA molecule of any one of claims 1-20, wherein the antisense strand comprises a structure represented by Formula I, wherein Formula I is, in the 5′-to-3′ direction:
A-B-(A′)ⱼ-C-P²-D-P¹—(C′-P¹)ₖ-C′ Formula I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

22. The siRNA molecule of claim 21, wherein the antisense strand comprises a structure represented by Formula A1, wherein Formula A1 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

23. The siRNA molecule of any one of claims 1-20, wherein the antisense strand comprises a structure represented by Formula II, wherein Formula II is, in the 5′-to-3′ direction:
A-B-(A′)ⱼ-C-P²-D-P¹—(C-P¹)ₖ-C′ Formula II;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

24. The siRNA molecule of claim 23, wherein the antisense strand comprises a structure represented by Formula A2, wherein Formula A2 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

25. The siRNA molecule of any one of claims 1-24, wherein the sense strand comprises a structure represented by Formula III, wherein Formula III is, in the 5′-to-3′ direction:
E-(A′)ₘ-F Formula III;
wherein E is represented by the formula (C-P¹)₂;
F is represented by the formula (C-P²)₃-D-P¹-C-P¹—C, (C-P²)₃-D-P²-C-P²—C, (C-P²)₃-D-P¹-C-P¹-D, or (C-P²)₃-D-P²-C-P²-D;
A′, C, D, P¹, and P² are as defined in Formula II; and
m is an integer from 1 to 7.

26. The siRNA molecule of claim 25, wherein the sense strand comprises a structure represented by Formula S1, wherein Formula S1 is, in the 5′-to-3′ direction:
A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

27. The siRNA molecule of claim 25, wherein the sense strand comprises a structure represented by Formula S2, wherein Formula S2 is, in the 5′-to-3′ direction:
A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

28. The siRNA molecule of claim 25, wherein the sense strand comprises a structure represented by Formula S3, wherein Formula S3 is, in the 5′-to-3′ direction:
A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

29. The siRNA molecule of claim 25, wherein the sense strand comprises a structure represented by Formula S4, wherein Formula S4 is, in the 5′-to-3′ direction:
A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

30. The siRNA molecule of any one of claims 1-20 and 25-29, wherein the antisense strand comprises a structure represented by Formula IV, wherein Formula IV is, in the 5′-to-3′ direction:
A-(A′)ⱼ-C-P²—B—(C-P¹)ₖ-C′ Formula IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

31. The siRNA molecule of claim 30, wherein the antisense strand comprises a structure represented by Formula A3, wherein Formula A3 is, in the 5′-to-3′ direction:
A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

32. The siRNA molecule of any one of claims 1-24, 30, and 31, wherein the sense strand comprises a structure represented by Formula V, wherein Formula V is, in the 5′-to-3′ direction:
E-(A′)ₘ-C-P²—F Formula V;
wherein E is represented by the formula (C-P¹)₂;
F is represented by the formula D-P¹-C-P¹—C, D-P²-C-P²—C, D-P¹-C-P¹-D, or D-P²-C-P²-D;
A′, C, D, P¹ and P² are as defined in Formula IV; and
m is an integer from 1 to 7.

33. The siRNA molecule of claim 32, wherein the sense strand comprises a structure represented by Formula S5, wherein Formula S5 is, in the 5′-to-3′ direction:
A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

34. The siRNA molecule of claim 32, wherein the sense strand comprises a structure represented by Formula S6, wherein Formula S6 is, in the 5′-to-3′ direction:
A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

35. The siRNA molecule of claim 32, wherein the sense strand comprises a structure represented by Formula S7, wherein Formula S7 is, in the 5′-to-3′ direction:
A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of claim 32, wherein the sense strand comprises a structure represented by Formula S8, wherein Formula S8 is, in the 5′-to-3′ direction:
A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

37. The siRNA molecule of any one of claims 1-20, 25-29 and 32-36, wherein the antisense strand comprises a structure represented by Formula VI, wherein Formula VI is, in the 5′-to-3′ direction:
A-Bⱼ-E-Bₖ-E-F-Gₗ-D-P¹—C′ Formula VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

38. The siRNA molecule of claim 37, wherein the antisense strand comprises a structure represented by Formula A4, wherein Formula A4 is, in the 5′-to-3′ direction:
A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

39. The siRNA molecule of any one of claims 1-24, 30, 31, 37, and 38, wherein the sense strand comprises a structure represented by Formula VII, wherein Formula VII is, in the 5′-to-3′ direction:
H-Bₘ-Iₙ-A′-Bₒ-H-C Formula VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7;
n is an integer from 1 to 7; and
is an integer from 1 to 7.

40. The siRNA molecule of claim 39, wherein the sense strand comprises a structure represented by Formula S9, wherein Formula S9 is, in the 5′-to-3′ direction:
A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of any one of claims 1-40, wherein the antisense strand further comprises a 5′ phosphorus stabilizing moiety at the 5′ end of the antisense strand.

42. The siRNA molecule of any one of claims 1-41, wherein the sense strand further comprises a 5′ phosphorus stabilizing moiety at the 5′ end of the sense strand.

43. The siRNA molecule of claim 41 or 42, wherein each 5′ phosphorus stabilizing moiety is, independently, represented by any one of Formulas IX-XVI: 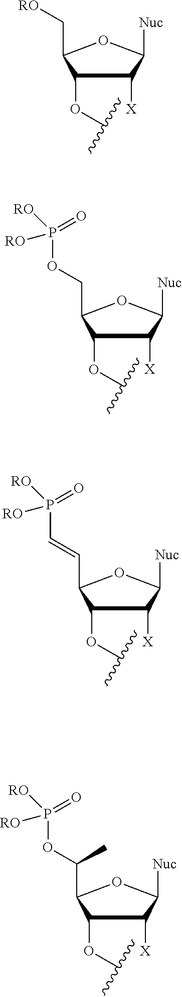 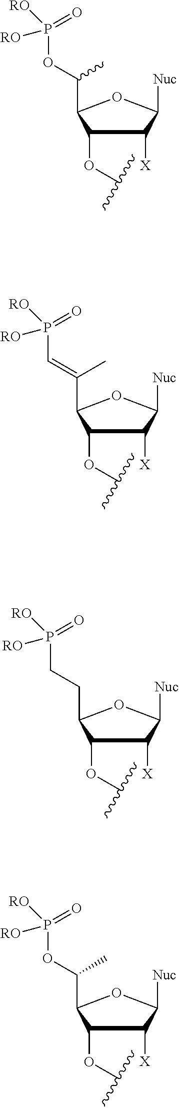
wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation, or hydrogen.

44. The siRNA molecule of claim 43, wherein the nucleobase is an adenine, uracil, guanine, thymine, or cytosine.

45. The siRNA molecule of any one of claims 41-44, wherein the 5′ phosphorus stabilizing moiety is (E)-vinylphosphonate represented by Formula XI.

46. The siRNA molecule of any one of claims 1-45, wherein the siRNA molecule further comprises a hydrophobic moiety at the 5′ or the 3′ end of the siRNA molecule.

47. The siRNA molecule of claim 46, wherein the hydrophobic moiety is selected from a group consisting of cholesterol, vitamin D, or tocopherol.

48. The siRNA molecule of any one of claims 1-47, wherein the length of the sense strand is between 10 and 30 nucleotides.

49. The siRNA molecule of claim 48, wherein the length of the sense strand is between 10 and 25 nucleotides.

50. The siRNA molecule of claim 49, wherein the length of the sense strand is between 12 and 25 nucleotides.

51. The siRNA molecule of claim 50, wherein the length of the sense strand is between 12 and 20 nucleotides.

52. The siRNA molecule of claim 51, wherein the length of the sense strand is between 12 and 19 nucleotides.

53. The siRNA molecule of claim 52, wherein the length of the sense strand is 15 nucleotides.

54. The siRNA molecule of claim 52, wherein the length of the sense strand is 16 nucleotides.

55. The siRNA molecule of claim 52, wherein the length of the sense strand is 18 nucleotides.

56. The siRNA molecule of any one of claims 1-55, wherein the length of the antisense strand is between 10 and 30 nucleotides.

57. The siRNA molecule of claim 56, wherein the length of the antisense strand is between 12 and 30 nucleotides.

58. The siRNA molecule of claim 57, wherein the length of the antisense strand is between 15 and 30 nucleotides.

59. The siRNA molecule of claim 58, wherein the length of the antisense strand is between 18 and 30 nucleotides.

60. The siRNA molecule of claim 59, wherein the length of the antisense strand is between 18 and 25 nucleotides.

61. The siRNA molecule of claim 60, wherein the length of the antisense strand is between 18 and 21 nucleotides.

62. The siRNA molecule of claim 61, wherein the length of the antisense strand is 18 nucleotides.

63. The siRNA molecule of claim 61, wherein the length of the antisense strand is 20 nucleotides.

64. The siRNA molecule of claim 61, wherein the length of the antisense strand is 21 nucleotides.

65. The siRNA molecule of any one of claims 1-64, wherein the siRNA molecule is a branched siRNA molecule.

66. The siRNA molecule of claim 65, wherein the branched siRNA molecule is di-branched, tri-branched, or tetra-branched.

67. The siRNA molecule of claim 66, wherein the siRNA molecule is a di-branched siRNA molecule, optionally wherein the di-branched siRNA molecule is represented by any one of Formulas XVI I-XIX: 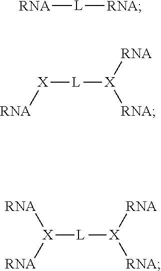
wherein each RNA is, independently, an siRNA molecule, L is a linker, and each X, independently, represents a branch point moiety.

68. The siRNA molecule of claim 66, wherein the siRNA molecule is a tri-branched siRNA molecule, optionally wherein the tri-branched siRNA molecule is represented by any one of Formulas XX-XXIII: 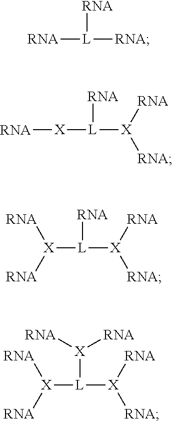
wherein each RNA is, independently, an siRNA molecule, L is a linker, and each X, independently, represents a branch point moiety.

69. The siRNA molecule of claim 66, wherein the siRNA molecule is a tetra-branched siRNA molecule, optionally wherein the tetra-branched siRNA molecule is represented by any one of Formulas XXIV-XXVIII: 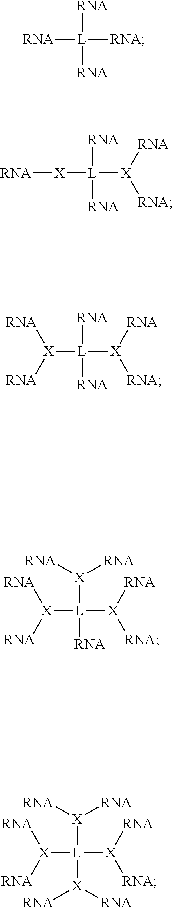
wherein each RNA is, independently, an siRNA molecule, L is a linker, and each X, independently, represents a branch point moiety.

70. The siRNA molecule of any one of claims 67-69, wherein the linker is selected from a group consisting of one or more contiguous subunits of an ethylene glycol, alkyl, carbohydrate, block copolymer, peptide, RNA, and DNA.

71. The siRNA molecule of claim 70, wherein the one or more contiguous subunits is 2 to 20 contiguous subunits.

72. A pharmaceutical composition comprising the siRNA molecule of any one of claims 1-71 and a pharmaceutically acceptable excipient, carrier, or diluent.

73. A method of delivering an siRNA molecule to a subject diagnosed as having a neuroinflammatory disease, the method comprising administering a therapeutically effective amount of the siRNA molecule of any one of claims 1-71 or the pharmaceutical composition of claim 72 to the subject.

74. A method of treating a neuroinflammatory disease in a subject in need thereof, the method comprising administering a therapeutically effective amount of the siRNA molecule of any one of claims 1-71 or the pharmaceutical composition of claim 72 to the subject.

75. The method of claim 73 or 74, wherein the neuroinflammatory disease is Alzheimer's disease.

76. A method of reducing MS4A6A expression in a subject in need thereof, the method comprising administering a therapeutically effective amount of the siRNA molecule of any one of claims 1-71 or the pharmaceutical composition of claim 72 to the subject.

77. The method of any one of claims 73-76, wherein the siRNA molecule or the pharmaceutical composition is administered to the subject by way of intracerebroventricular, intrastriatal, intraparenchymal, or intrathecal injection.

78. The method of any one of claims 73-76, wherein the siRNA molecule or the pharmaceutical composition is administered to the subject by way of intravenous, intramuscular, or subcutaneous injection.

79. The method of any one of claims 73-78, wherein the subject is a human.

80. A kit comprising the siRNA molecule of any one of claims 1-71, or the pharmaceutical composition of claim 72, and a package insert, wherein the package insert instructs a user of the kit to perform the method of any one of claims 73-79.


SEQUENCE LISTING (576 sequences)

SEQ ID NO: 1 - RNA, 21 nt, Homo sapiens
  source 1..21: mol_type = mRNA
gatgactttcagaagatgaag

SEQ ID NO: 2 - RNA, 21 nt, Homo sapiens
  source 1..21: mol_type = mRNA
atgactttcagaagatgaagg

SEQ ID NO: 3 - RNA, 21 nt, Homo sapiens
  source 1..21: mol_type = mRNA
tgactttcagaagatgaaggt

SEQ ID NO: 4 - RNA, 21 nt, Homo sapiens
  source 1..21: mol_type = mRNA
ttcagaagatgaaggtaagta

SEQ ID NO: 5 - RNA, 21 nt, Homo sapiens
  source 1..21: mol_type = mRNA
tcagaagatgaaggtaagtag

SEQ ID NO: 6 - RNA, 21 nt, Homo sapiens
  source 1..21: mol_type = mRNA
cagaagatgaaggtaagtaga

SEQ ID NO: 7 - RNA, 21 nt, Homo sapiens
  source 1..21: mol_type = mRNA
agaagatgaaggtaagtagaa

SEQ ID NO: 8 - RNA, 21 nt, Homo sapiens
  source 1..21: mol_type = mRNA
gaagatgaaggtaagtagaaa

SEQ ID NO: 9 - RNA, 21 nt, Homo sapiens
  source 1..21: mol_type = mRNA
gatgaaggtaagtagaaaccg

SEQ ID NO: 10 - RNA, 21 nt, Homo sapiens
  source 1..21: mol_type = mRNA
accgttgatgggactgagaaa

SEQ ID NO: 11 - RNA, 21 nt, Homo sapiens
  source 1..21: mol_type = mRNA
gaaaccagagttaaaacctct

SEQ ID NO: 12 - RNA, 21 nt, Homo sapiens
  source 1..21: mol_type = mRNA
aaccagagttaaaacctcttt

SEQ ID NO: 13 - RNA, 21 nt, Homo sapiens
  source 1..21: mol_type = mRNA
tttggagcttctgaggactca

SEQ ID NO: 14 - RNA, 21 nt, Homo sapiens
  source 1..21: mol_type = mRNA
ttggagcttctgaggactcag

SEQ ID NO: 15 - RNA, 21 nt, Homo sapiens
  source 1..21: mol_type = mRNA
cgggcacagttggcaacacca

SEQ ID NO: 16 - RNA, 21 nt, Homo sapiens
  source 1..21: mol_type = mRNA
ggcacagttggcaacaccatc

SEQ ID NO: 17 - RNA, 21 nt, Homo sapiens
  source 1..21: mol_type = mRNA
gcacagttggcaacaccatca

SEQ ID NO: 18 - RNA, 21 nt, Homo sapiens
  source 1..21: mol_type = mRNA
cacagttggcaacaccatcat

SEQ ID NO: 19 - RNA, 21 nt, Homo sapiens
  source 1..21: mol_type = mRNA
agttggcaacaccatcatgac

SEQ ID NO: 20 - RNA, 21 nt, Homo sapiens
  source 1..21: mol_type = mRNA
gttggcaacaccatcatgaca

SEQ ID NO: 21 - RNA, 21 nt, Homo sapiens
  source 1..21: mol_type = mRNA
caacaccatcatgacatcaca

SEQ ID NO: 22 - RNA, 21 nt, Homo sapiens
  source 1..21: mol_type = mRNA
aacaccatcatgacatcacaa

SEQ ID NO: 23 - RNA, 21 nt, Homo sapiens
  source 1..21: mol_type = mRNA
acaccatcatgacatcacaac

SEQ ID NO: 24 - RNA, 21 nt, Homo sapiens
  source 1..21: mol_type = mRNA
tcatgacatcacaacctgttc

SEQ ID NO: 25 - RNA, 21 nt, Homo sapiens
  source 1..21: mol_type = mRNA
catgacatcacaacctgttcc

SEQ ID NO: 26 - RNA, 21 nt, Homo sapiens
  source 1..21: mol_type = mRNA
atgacatcacaacctgttccc

SEQ ID NO: 27 - RNA, 21 nt, Homo sapiens
  source 1..21: mol_type = mRNA
tgacatcacaacctgttccca

SEQ ID NO: 28 - RNA, 21 nt, Homo sapiens
  source 1..21: mol_type = mRNA
acatcacaacctgttcccaat

SEQ ID NO: 29 - RNA, 21 nt, Homo sapiens
  source 1..21: mol_type = mRNA
catcacaacctgttcccaatg

SEQ ID NO: 30 - RNA, 21 nt, Homo sapiens
  source 1..21: mol_type = mRNA
tcacaacctgttcccaatgag

SEQ ID NO: 31 - RNA, 21 nt, Homo sapiens
  source 1..21: mol_type = mRNA
cacaacctgttcccaatgaga

SEQ ID NO: 32 - RNA, 21 nt, Homo sapiens
  source 1..21: mol_type = mRNA
caacctgttcccaatgagacc

SEQ ID NO: 33 - RNA, 21 nt, Homo sapiens
  source 1..21: mol_type = mRNA
catcatagtgctcccatcaaa

SEQ ID NO: 34 - RNA, 21 nt, Homo sapiens
  source 1..21: mol_type = mRNA
atcatagtgctcccatcaaat

SEQ ID NO: 35 - RNA, 21 nt, Homo sapiens
  source 1..21: mol_type = mRNA
tcatagtgctcccatcaaatg

SEQ ID NO: 36 - RNA, 21 nt, Homo sapiens
  source 1..21: mol_type = mRNA
catagtgctcccatcaaatgt

SEQ ID NO: 37 - RNA, 21 nt, Homo sapiens
  source 1..21: mol_type = mRNA
aatcaaagttattgggactat

SEQ ID NO: 38 - RNA, 21 nt, Homo sapiens
  source 1..21: mol_type = mRNA
atcaaagttattgggactatc

SEQ ID NO: 39 - RNA, 21 nt, Homo sapiens
  source 1..21: mol_type = mRNA
tcaaagttattgggactatcc

SEQ ID NO: 40 - RNA, 21 nt, Homo sapiens
  source 1..21: mol_type = mRNA
caaagttattgggactatcca

SEQ ID NO: 41 - RNA, 21 nt, Homo sapiens
  source 1..21: mol_type = mRNA
aaagttattgggactatccag

SEQ ID NO: 42 - RNA, 21 nt, Homo sapiens
  source 1..21: mol_type = mRNA
aagttattgggactatccaga

SEQ ID NO: 43 - RNA, 21 nt, Homo sapiens
  source 1..21: mol_type = mRNA
agttattgggactatccagat

SEQ ID NO: 44 - RNA, 21 nt, Homo sapiens
  source 1..21: mol_type = mRNA
gttattgggactatccagatc

SEQ ID NO: 45 - RNA, 21 nt, Homo sapiens
  source 1..21: mol_type = mRNA
ttattgggactatccagatct

SEQ ID NO: 46 - RNA, 21 nt, Homo sapiens
  source 1..21: mol_type = mRNA
tattgggactatccagatctt

SEQ ID NO: 47 - RNA, 21 nt, Homo sapiens
  source 1..21: mol_type = mRNA
attgggactatccagatcttg

SEQ ID NO: 48 - RNA, 21 nt, Homo sapiens
  source 1..21: mol_type = mRNA
ttgggactatccagatcttgt

SEQ ID NO: 49 - RNA, 21 nt, Homo sapiens
  source 1..21: mol_type = mRNA
tgggactatccagatcttgtg

SEQ ID NO: 50 - RNA, 21 nt, Homo sapiens
  source 1..21: mol_type = mRNA
ggactatccagatcttgtgtg

SEQ ID NO: 51 - RNA, 21 nt, Homo sapiens
  source 1..21: mol_type = mRNA
actatccagatcttgtgtggc

SEQ ID NO: 52 - RNA, 21 nt, Homo sapiens
  source 1..21: mol_type = mRNA
attttggcatctgcttccttc

SEQ ID NO: 53 - RNA, 21 nt, Homo sapiens
  source 1..21: mol_type = mRNA
ttttggcatctgcttccttct

SEQ ID NO: 54 - RNA, 21 nt, Homo sapiens
  source 1..21: mol_type = mRNA
ttggcatctgcttccttctct

SEQ ID NO: 55 - RNA, 21 nt, Homo sapiens
  source 1..21: mol_type = mRNA
tggcatctgcttccttctctc

SEQ ID NO: 56 - RNA, 21 nt, Homo sapiens
  source 1..21: mol_type = mRNA
gcatctgcttccttctctcca

SEQ ID NO: 57 - RNA, 21 nt, Homo sapiens
  source 1..21: mol_type = mRNA
catctgcttccttctctccaa

SEQ ID NO: 58 - RNA, 21 nt, Homo sapiens
  source 1..21: mol_type = mRNA
gcttccttctctccaaatttt

SEQ ID NO: 59 - RNA, 21 nt, Homo sapiens
  source 1..21: mol_type = mRNA
cttccttctctccaaatttta

SEQ ID NO: 60 - RNA, 21 nt, Homo sapiens
  source 1..21: mol_type = mRNA
ttccttctctccaaattttac

SEQ ID NO: 61 - RNA, 21 nt, Homo sapiens
  source 1..21: mol_type = mRNA
tccttctctccaaattttacc

SEQ ID NO: 62 - RNA, 21 nt, Homo sapiens
  source 1..21: mol_type = mRNA
ccttctctccaaattttaccc

SEQ ID NO: 63 - RNA, 21 nt, Homo sapiens
  source 1..21: mol_type = mRNA
cttctctccaaattttaccca

SEQ ID NO: 64 - RNA, 21 nt, Homo sapiens
  source 1..21: mol_type = mRNA
ttctctccaaattttacccaa

SEQ ID NO: 65 - RNA, 21 nt, Homo sapiens
  source 1..21: mol_type = mRNA
tctctccaaattttacccaag

SEQ ID NO: 66 - RNA, 21 nt, Homo sapiens
  source 1..21: mol_type = mRNA
tctccaaattttacccaagtg

SEQ ID NO: 67 - RNA, 21 nt, Homo sapiens
  source 1..21: mol_type = mRNA
ctccaaattttacccaagtga

SEQ ID NO: 68 - RNA, 21 nt, Homo sapiens
  source 1..21: mol_type = mRNA
tccaaattttacccaagtgac

SEQ ID NO: 69 - RNA, 21 nt, Homo sapiens
  source 1..21: mol_type = mRNA
caaattttacccaagtgactt

SEQ ID NO: 70 - RNA, 21 nt, Homo sapiens
  source 1..21: mol_type = mRNA
aaattttacccaagtgacttc

SEQ ID NO: 71 - RNA, 21 nt, Homo sapiens
  source 1..21: mol_type = mRNA
aattttacccaagtgacttct

SEQ ID NO: 72 - RNA, 21 nt, Homo sapiens
  source 1..21: mol_type = mRNA
attttacccaagtgacttcta

SEQ ID NO: 73 - RNA, 21 nt, Homo sapiens
  source 1..21: mol_type = mRNA
ttttacccaagtgacttctac

SEQ ID NO: 74 - RNA, 21 nt, Homo sapiens
  source 1..21: mol_type = mRNA
tttacccaagtgacttctaca

SEQ ID NO: 75 - RNA, 21 nt, Homo sapiens
  source 1..21: mol_type = mRNA
ttacccaagtgacttctacac

SEQ ID NO: 76 - RNA, 21 nt, Homo sapiens
  source 1..21: mol_type = mRNA
tacccaagtgacttctacact

SEQ ID NO: 77 - RNA, 21 nt, Homo sapiens
  source 1..21: mol_type = mRNA
acccaagtgacttctacactg

SEQ ID NO: 78 - RNA, 21 nt, Homo sapiens
  source 1..21: mol_type = mRNA
ccaagtgacttctacactgtt

SEQ ID NO: 79 - RNA, 21 nt, Homo sapiens
  source 1..21: mol_type = mRNA
caagtgacttctacactgttg

SEQ ID NO: 80 - RNA, 21 nt, Homo sapiens
  source 1..21: mol_type = mRNA
aagtgacttctacactgttga

SEQ ID NO: 81 - RNA, 21 nt, Homo sapiens
  source 1..21: mol_type = mRNA
tgacttctacactgttgaact

SEQ ID NO: 82 - RNA, 21 nt, Homo sapiens
  source 1..21: mol_type = mRNA
gacttctacactgttgaactc

SEQ ID NO: 83 - RNA, 21 nt, Homo sapiens
  source 1..21: mol_type = mRNA
cttctacactgttgaactctg

SEQ ID NO: 84 - RNA, 21 nt, Homo sapiens
  source 1..21: mol_type = mRNA
tctacactgttgaactctgct

SEQ ID NO: 85 - RNA, 21 nt, Homo sapiens
  source 1..21: mol_type = mRNA
ctacactgttgaactctgctt

SEQ ID NO: 86 - RNA, 21 nt, Homo sapiens
  source 1..21: mol_type = mRNA
tacactgttgaactctgctta

SEQ ID NO: 87 - RNA, 21 nt, Homo sapiens
  source 1..21: mol_type = mRNA
cactgttgaactctgcttacc

SEQ ID NO: 88 - RNA, 21 nt, Homo sapiens
  source 1..21: mol_type = mRNA
actgttgaactctgcttaccc

SEQ ID NO: 89 - RNA, 21 nt, Homo sapiens
  source 1..21: mol_type = mRNA
ctgttgaactctgcttaccca

SEQ ID NO: 90 - RNA, 21 nt, Homo sapiens
  source 1..21: mol_type = mRNA
tgttgaactctgcttacccat

SEQ ID NO: 91 - RNA, 21 nt, Homo sapiens
  source 1..21: mol_type = mRNA
gttgaactctgcttacccatt

SEQ ID NO: 92 - RNA, 21 nt, Homo sapiens
  source 1..21: mol_type = mRNA
tgaactctgcttacccattca

SEQ ID NO: 93 - RNA, 21 nt, Homo sapiens
  source 1..21: mol_type = mRNA
aactctgcttacccattcata

SEQ ID NO: 94 - RNA, 21 nt, Homo sapiens
  source 1..21: mol_type = mRNA
actctgcttacccattcatag

SEQ ID NO: 95 - RNA, 21 nt, Homo sapiens
  source 1..21: mol_type = mRNA
ctctgcttacccattcatagg

SEQ ID NO: 96 - RNA, 21 nt, Homo sapiens
  source 1..21: mol_type = mRNA
tctgcttacccattcatagga

SEQ ID NO: 97 - RNA, 21 nt, Homo sapiens
  source 1..21: mol_type = mRNA
tgcttacccattcataggacc

SEQ ID NO: 98 - RNA, 21 nt, Homo sapiens
  source 1..21: mol_type = mRNA
gcttacccattcataggaccc

SEQ ID NO: 99 - RNA, 21 nt, Homo sapiens
  source 1..21: mol_type = mRNA
cttacccattcataggaccct

SEQ ID NO: 100 - RNA, 21 nt, Homo sapiens
  source 1..21: mol_type = mRNA
ttacccattcataggaccctt

SEQ ID NO: 101 - RNA, 21 nt, Homo sapiens
  source 1..21: mol_type = mRNA
tacccattcataggacccttt

SEQ ID NO: 102 - RNA, 21 nt, Homo sapiens
  source 1..21: mol_type = mRNA
acccattcataggaccctttt

SEQ ID NO: 103 - RNA, 21 nt, Homo sapiens
  source 1..21: mol_type = mRNA
cccattcataggacccttttt

SEQ ID NO: 104 - RNA, 21 nt, Homo sapiens
  source 1..21: mol_type = mRNA
ccattcataggaccctttttt

SEQ ID NO: 105 - RNA, 21 nt, Homo sapiens
  source 1..21: mol_type = mRNA
cataggaccctttttttttat

SEQ ID NO: 106 - RNA, 21 nt, Homo sapiens
  source 1..21: mol_type = mRNA
taggaccctttttttttatca

SEQ ID NO: 107 - RNA, 21 nt, Homo sapiens
  source 1..21: mol_type = mRNA
catctctggctctctatcaat

SEQ ID NO: 108 - RNA, 21 nt, Homo sapiens
  source 1..21: mol_type = mRNA
atctctggctctctatcaatc

SEQ ID NO: 109 - RNA, 21 nt, Homo sapiens
  source 1..21: mol_type = mRNA
gttaaccaagcttttggtgca

SEQ ID NO: 110 - RNA, 21 nt, Homo sapiens
  source 1..21: mol_type = mRNA
taaccaagcttttggtgcata

SEQ ID NO: 111 - RNA, 21 nt, Homo sapiens
  source 1..21: mol_type = mRNA
aaccaagcttttggtgcatag

SEQ ID NO: 112 - RNA, 21 nt, Homo sapiens
  source 1..21: mol_type = mRNA
accaagcttttggtgcatagc

SEQ ID NO: 113 - RNA, 21 nt, Homo sapiens
  source 1..21: mol_type = mRNA
ccaagcttttggtgcatagca

SEQ ID NO: 114 - RNA, 21 nt, Homo sapiens
  source 1..21: mol_type = mRNA
caagcttttggtgcatagcag

SEQ ID NO: 115 - RNA, 21 nt, Homo sapiens
  source 1..21: mol_type = mRNA
aagcttttggtgcatagcagc

SEQ ID NO: 116 - RNA, 21 nt, Homo sapiens
  source 1..21: mol_type = mRNA
agcttttggtgcatagcagcc

SEQ ID NO: 117 - RNA, 21 nt, Homo sapiens
  source 1..21: mol_type = mRNA
gcttttggtgcatagcagcct

SEQ ID NO: 118 - RNA, 21 nt, Homo sapiens
  source 1..21: mol_type = mRNA
ttggtgcatagcagcctggtt

SEQ ID NO: 119 - RNA, 21 nt, Homo sapiens
  source 1..21: mol_type = mRNA
tggtgcatagcagcctggttg

SEQ ID NO: 120 - RNA, 21 nt, Homo sapiens
  source 1..21: mol_type = mRNA
agcagcctggttggaagcatt

SEQ ID NO: 121 - RNA, 21 nt, Homo sapiens
  source 1..21: mol_type = mRNA
gcagcctggttggaagcattc

SEQ ID NO: 122 - RNA, 21 nt, Homo sapiens
  source 1..21: mol_type = mRNA
cagcctggttggaagcattct

SEQ ID NO: 123 - RNA, 21 nt, Homo sapiens
  source 1..21: mol_type = mRNA
agcctggttggaagcattctg

SEQ ID NO: 124 - RNA, 21 nt, Homo sapiens
  source 1..21: mol_type = mRNA
gcctggttggaagcattctga

SEQ ID NO: 125 - RNA, 21 nt, Homo sapiens
  source 1..21: mol_type = mRNA
ctggttggaagcattctgagt

SEQ ID NO: 126 - RNA, 21 nt, Homo sapiens
  source 1..21: mol_type = mRNA
gccctggtgggtttcattatc

SEQ ID NO: 127 - RNA, 21 nt, Homo sapiens
  source 1..21: mol_type = mRNA
ccctggtgggtttcattatcc

SEQ ID NO: 128 - RNA, 21 nt, Homo sapiens
  source 1..21: mol_type = mRNA
cctggtgggtttcattatcct

SEQ ID NO: 129 - RNA, 21 nt, Homo sapiens
  source 1..21: mol_type = mRNA
ctggtgggtttcattatcctg

SEQ ID NO: 130 - RNA, 21 nt, Homo sapiens
  source 1..21: mol_type = mRNA
tggtgggtttcattatcctgt

SEQ ID NO: 131 - RNA, 21 nt, Homo sapiens
  source 1..21: mol_type = mRNA
gggtttcattatcctgtctgt

SEQ ID NO: 132 - RNA, 21 nt, Homo sapiens
  source 1..21: mol_type = mRNA
gtttcattatcctgtctgtca

SEQ ID NO: 133 - RNA, 21 nt, Homo sapiens
  source 1..21: mol_type = mRNA
ctgcagtgtgagttggacaaa

SEQ ID NO: 134 - RNA, 21 nt, Homo sapiens
  source 1..21: mol_type = mRNA
tgcagtgtgagttggacaaaa

SEQ ID NO: 135 - RNA, 21 nt, Homo sapiens
  source 1..21: mol_type = mRNA
gcagtgtgagttggacaaaaa

SEQ ID NO: 136 - RNA, 21 nt, Homo sapiens
  source 1..21: mol_type = mRNA
cagtgtgagttggacaaaaat

SEQ ID NO: 137 - RNA, 21 nt, Homo sapiens
  source 1..21: mol_type = mRNA
agtgtgagttggacaaaaata

SEQ ID NO: 138 - RNA, 21 nt, Homo sapiens
  source 1..21: mol_type = mRNA
gtgtgagttggacaaaaataa

SEQ ID NO: 139 - RNA, 21 nt, Homo sapiens
  source 1..21: mol_type = mRNA
tgtgagttggacaaaaataat

SEQ ID NO: 140 - RNA, 21 nt, Homo sapiens
  source 1..21: mol_type = mRNA
gtgagttggacaaaaataata

SEQ ID NO: 141 - RNA, 21 nt, Homo sapiens
  source 1..21: mol_type = mRNA
taccacggactgctatacagc

SEQ ID NO: 142 - RNA, 21 nt, Homo sapiens
  source 1..21: mol_type = mRNA
tctgatgctgatttgcactct

SEQ ID NO: 143 - RNA, 21 nt, Homo sapiens
  source 1..21: mol_type = mRNA
ctgatgctgatttgcactctg

SEQ ID NO: 144 - RNA, 21 nt, Homo sapiens
  source 1..21: mol_type = mRNA
ctgatttgcactctgctggaa

SEQ ID NO: 145 - RNA, 21 nt, Homo sapiens
  source 1..21: mol_type = mRNA
tgatttgcactctgctggaat

SEQ ID NO: 146 - RNA, 21 nt, Homo sapiens
  source 1..21: mol_type = mRNA
tttgcactctgctggaattct

SEQ ID NO: 147 - RNA, 21 nt, Homo sapiens
  source 1..21: mol_type = mRNA
ttgcactctgctggaattctg

SEQ ID NO: 148 - RNA, 21 nt, Homo sapiens
  source 1..21: mol_type = mRNA
ctctgctggaattctgcctag

SEQ ID NO: 149 - RNA, 21 nt, Homo sapiens
  source 1..21: mol_type = mRNA
tgctggaattctgcctagctg

SEQ ID NO: 150 - RNA, 21 nt, Homo sapiens
  source 1..21: mol_type = mRNA
gctggaattctgcctagctgt

SEQ ID NO: 151 - RNA, 21 nt, Homo sapiens
  source 1..21: mol_type = mRNA
ctggaattctgcctagctgtg

SEQ ID NO: 152 - RNA, 21 nt, Homo sapiens
  source 1..21: mol_type = mRNA
tggaattctgcctagctgtgc

SEQ ID NO: 153 - RNA, 21 nt, Homo sapiens
  source 1..21: mol_type = mRNA
ctgcggtggaaacaggcttac

SEQ ID NO: 154 - RNA, 21 nt, Homo sapiens
  source 1..21: mol_type = mRNA
tgcggtggaaacaggcttact

SEQ ID NO: 155 - RNA, 21 nt, Homo sapiens
  source 1..21: mol_type = mRNA
gcggtggaaacaggcttactc

SEQ ID NO: 156 - RNA, 21 nt, Homo sapiens
  source 1..21: mol_type = mRNA
cggtggaaacaggcttactct

SEQ ID NO: 157 - RNA, 21 nt, Homo sapiens
  source 1..21: mol_type = mRNA
tggaaacaggcttactctgac

SEQ ID NO: 158 - RNA, 21 nt, Homo sapiens
  source 1..21: mol_type = mRNA
ggaaacaggcttactctgact

SEQ ID NO: 159 - RNA, 21 nt, Homo sapiens
  source 1..21: mol_type = mRNA
gaaacaggcttactctgactt

SEQ ID NO: 160 - RNA, 21 nt, Homo sapiens
  source 1..21: mol_type = mRNA
aaacaggcttactctgacttc

SEQ ID NO: 161 - RNA, 21 nt, Homo sapiens
  source 1..21: mol_type = mRNA
aacaggcttactctgacttcc

SEQ ID NO: 162 - RNA, 21 nt, Homo sapiens
  source 1..21: mol_type = mRNA
acaggcttactctgacttccc

SEQ ID NO: 163 - RNA, 21 nt, Homo sapiens
  source 1..21: mol_type = mRNA
caggcttactctgacttccct

SEQ ID NO: 164 - RNA, 21 nt, Homo sapiens
  source 1..21: mol_type = mRNA
aggcttactctgacttccctg

SEQ ID NO: 165 - RNA, 21 nt, Homo sapiens
  source 1..21: mol_type = mRNA
ggcttactctgacttccctgg

SEQ ID NO: 166 - RNA, 21 nt, Homo sapiens
  source 1..21: mol_type = mRNA
tgtacttttcctgcctcacag

SEQ ID NO: 167 - RNA, 21 nt, Homo sapiens
  source 1..21: mol_type = mRNA
cttttcctgcctcacagttac

SEQ ID NO: 168 - RNA, 21 nt, Homo sapiens
  source 1..21: mol_type = mRNA
ttttcctgcctcacagttaca

SEQ ID NO: 169 - RNA, 21 nt, Homo sapiens
  source 1..21: mol_type = mRNA
tttcctgcctcacagttacat

SEQ ID NO: 170 - RNA, 21 nt, Homo sapiens
  source 1..21: mol_type = mRNA
gaagaactattgacttcttaa

SEQ ID NO: 171 - RNA, 21 nt, Homo sapiens
  source 1..21: mol_type = mRNA
agaactattgacttcttaaga

SEQ ID NO: 172 - RNA, 21 nt, Homo sapiens
  source 1..21: mol_type = mRNA
gaactattgacttcttaagaa

SEQ ID NO: 173 - RNA, 21 nt, Homo sapiens
  source 1..21: mol_type = mRNA
tgacttcttaagaaaaaaggg

SEQ ID NO: 174 - RNA, 21 nt, Homo sapiens
  source 1..21: mol_type = mRNA
gacttcttaagaaaaaaggga

SEQ ID NO: 175 - RNA, 21 nt, Homo sapiens
  source 1..21: mol_type = mRNA
acttcttaagaaaaaagggag

SEQ ID NO: 176 - RNA, 21 nt, Homo sapiens
  source 1..21: mol_type = mRNA
cttcttaagaaaaaagggaga

SEQ ID NO: 177 - RNA, 21 nt, Homo sapiens
  source 1..21: mol_type = mRNA
ttcttaagaaaaaagggagaa

SEQ ID NO: 178 - RNA, 21 nt, Homo sapiens
  source 1..21: mol_type = mRNA
cttaagaaaaaagggagaaat

SEQ ID NO: 179 - RNA, 21 nt, Homo sapiens
  source 1..21: mol_type = mRNA
aagggagaaatattaatcaga

SEQ ID NO: 180 - RNA, 21 nt, Homo sapiens
  source 1..21: mol_type = mRNA
agggagaaatattaatcagaa

SEQ ID NO: 181 - RNA, 21 nt, Homo sapiens
  source 1..21: mol_type = mRNA
gggagaaatattaatcagaaa

SEQ ID NO: 182 - RNA, 21 nt, Homo sapiens
  source 1..21: mol_type = mRNA
tcagaaagttgattcttatga

SEQ ID NO: 183 - RNA, 21 nt, Homo sapiens
  source 1..21: mol_type = mRNA
cagaaagttgattcttatgat

SEQ ID NO: 184 - RNA, 21 nt, Homo sapiens
  source 1..21: mol_type = mRNA
atagaaaagcaaagcttgagt

SEQ ID NO: 185 - RNA, 21 nt, Homo sapiens
  source 1..21: mol_type = mRNA
tagaaaagcaaagcttgagtt

SEQ ID NO: 186 - RNA, 21 nt, Homo sapiens
  source 1..21: mol_type = mRNA
agaaaagcaaagcttgagttt

SEQ ID NO: 187 - RNA, 21 nt, Homo sapiens
  source 1..21: mol_type = mRNA
gaaaagcaaagcttgagtttc

SEQ ID NO: 188 - RNA, 21 nt, Homo sapiens
  source 1..21: mol_type = mRNA
aaaagcaaagcttgagtttcc

SEQ ID NO: 189 - RNA, 21 nt, Homo sapiens
  source 1..21: mol_type = mRNA
aaagcaaagcttgagtttcct

SEQ ID NO: 190 - RNA, 21 nt, Homo sapiens
  source 1..21: mol_type = mRNA
agcaaagcttgagtttcctaa

SEQ ID NO: 191 - RNA, 21 nt, Homo sapiens
  source 1..21: mol_type = mRNA
gcaaagcttgagtttcctaaa

SEQ ID NO: 192 - RNA, 21 nt, Homo sapiens
  source 1..21: mol_type = mRNA
caaagcttgagtttcctaaat

SEQ ID NO: 193 - RNA, 16 nt, Synthetic construct
  source 1..16: mol_type = other RNA
ctttcagaagatgaaa

SEQ ID NO: 194 - RNA, 16 nt, Synthetic construct
  source 1..16: mol_type = other RNA
tttcagaagatgaaga

SEQ ID NO: 195 - RNA, 16 nt, Synthetic construct
  source 1..16: mol_type = other RNA
ttcagaagatgaagga

SEQ ID NO: 196 - RNA, 16 nt, Synthetic construct
  source 1..16: mol_type = other RNA
aagatgaaggtaagta

SEQ ID NO: 197 - RNA, 16 nt, Synthetic construct
  source 1..16: mol_type = other RNA
agatgaaggtaagtaa

SEQ ID NO: 198 - RNA, 16 nt, Synthetic construct
  source 1..16: mol_type = other RNA
gatgaaggtaagtaga

SEQ ID NO: 199 - RNA, 16 nt, Synthetic construct
  source 1..16: mol_type = other RNA
atgaaggtaagtagaa

SEQ ID NO: 200 - RNA, 16 nt, Synthetic construct
  source 1..16: mol_type = other RNA
tgaaggtaagtagaaa

SEQ ID NO: 201 - RNA, 16 nt, Synthetic construct
  source 1..16: mol_type = other RNA
aggtaagtagaaacca

SEQ ID NO: 202 - RNA, 16 nt, Synthetic construct
  source 1..16: mol_type = other RNA
tgatgggactgagaaa

SEQ ID NO: 203 - RNA, 16 nt, Synthetic construct
  source 1..16: mol_type = other RNA
cagagttaaaacctca

SEQ ID NO: 204 - RNA, 16 nt, Synthetic construct
  source 1..16: mol_type = other RNA
gagttaaaacctctta

SEQ ID NO: 205 - RNA, 16 nt, Synthetic construct
  source 1..16: mol_type = other RNA
agcttctgaggactca

SEQ ID NO: 206 - RNA, 16 nt, Synthetic construct
  source 1..16: mol_type = other RNA
gcttctgaggactcaa

SEQ ID NO: 207 - RNA, 16 nt, Synthetic construct
  source 1..16: mol_type = other RNA
acagttggcaacacca

SEQ ID NO: 208 - RNA, 16 nt, Synthetic construct
  source 1..16: mol_type = other RNA
agttggcaacaccata

SEQ ID NO: 209 - RNA, 16 nt, Synthetic construct
  source 1..16: mol_type = other RNA
gttggcaacaccatca

SEQ ID NO: 210 - RNA, 16 nt, Synthetic construct
  source 1..16: mol_type = other RNA
ttggcaacaccatcaa

SEQ ID NO: 211 - RNA, 16 nt, Synthetic construct
  source 1..16: mol_type = other RNA
gcaacaccatcatgaa

SEQ ID NO: 212 - RNA, 16 nt, Synthetic construct
  source 1..16: mol_type = other RNA
caacaccatcatgaca

SEQ ID NO: 213 - RNA, 16 nt, Synthetic construct
  source 1..16: mol_type = other RNA
ccatcatgacatcaca

SEQ ID NO: 214 - RNA, 16 nt, Synthetic construct
  source 1..16: mol_type = other RNA
catcatgacatcacaa

SEQ ID NO: 215 - RNA, 16 nt, Synthetic construct
  source 1..16: mol_type = other RNA
atcatgacatcacaaa

SEQ ID NO: 216 - RNA, 16 nt, Synthetic construct
  source 1..16: mol_type = other RNA
acatcacaacctgtta

SEQ ID NO: 217 - RNA, 16 nt, Synthetic construct
  source 1..16: mol_type = other RNA
catcacaacctgttca

SEQ ID NO: 218 - RNA, 16 nt, Synthetic construct
  source 1..16: mol_type = other RNA
atcacaacctgttcca

SEQ ID NO: 219 - RNA, 16 nt, Synthetic construct
  source 1..16: mol_type = other RNA
tcacaacctgttccca

SEQ ID NO: 220 - RNA, 16 nt, Synthetic construct
  source 1..16: mol_type = other RNA
acaacctgttcccaaa

SEQ ID NO: 221 - RNA, 16 nt, Synthetic construct
  source 1..16: mol_type = other RNA
caacctgttcccaata

SEQ ID NO: 222 - RNA, 16 nt, Synthetic construct
  source 1..16: mol_type = other RNA
acctgttcccaatgaa

SEQ ID NO: 223 - RNA, 16 nt, Synthetic construct
  source 1..16: mol_type = other RNA
cctgttcccaatgaga

SEQ ID NO: 224 - RNA, 16 nt, Synthetic construct
  source 1..16: mol_type = other RNA
tgttcccaatgagaca

SEQ ID NO: 225 - RNA, 16 nt, Synthetic construct
  source 1..16: mol_type = other RNA
tagtgctcccatcaaa

SEQ ID NO: 226 - RNA, 16 nt, Synthetic construct
  source 1..16: mol_type = other RNA
agtgctcccatcaaaa

SEQ ID NO: 227 - RNA, 16 nt, Synthetic construct
  source 1..16: mol_type = other RNA
gtgctcccatcaaata

SEQ ID NO: 228 - RNA, 16 nt, Synthetic construct
  source 1..16: mol_type = other RNA
tgctcccatcaaatga

SEQ ID NO: 229 - RNA, 16 nt, Synthetic construct
  source 1..16: mol_type = other RNA
aagttattgggactaa

SEQ ID NO: 230 - RNA, 16 nt, Synthetic construct
  source 1..16: mol_type = other RNA
agttattgggactata

SEQ ID NO: 231 - RNA, 16 nt, Synthetic construct
  source 1..16: mol_type = other RNA
gttattgggactatca

SEQ ID NO: 232 - RNA, 16 nt, Synthetic construct
  source 1..16: mol_type = other RNA
ttattgggactatcca

SEQ ID NO: 233 - RNA, 16 nt, Synthetic construct
  source 1..16: mol_type = other RNA
tattgggactatccaa

SEQ ID NO: 234 - RNA, 16 nt, Synthetic construct
  source 1..16: mol_type = other RNA
attgggactatccaga

SEQ ID NO: 235 - RNA, 16 nt, Synthetic construct
  source 1..16: mol_type = other RNA
ttgggactatccagaa

SEQ ID NO: 236 - RNA, 16 nt, Synthetic construct
  source 1..16: mol_type = other RNA
tgggactatccagata

SEQ ID NO: 237 - RNA, 16 nt, Synthetic construct
  source 1..16: mol_type = other RNA
gggactatccagatca

SEQ ID NO: 238 - RNA, 16 nt, Synthetic construct
  source 1..16: mol_type = other RNA
ggactatccagatcta

SEQ ID NO: 239 - RNA, 16 nt, Synthetic construct
  source 1..16: mol_type = other RNA
gactatccagatctta

SEQ ID NO: 240 - RNA, 16 nt, Synthetic construct
  source 1..16: mol_type = other RNA
actatccagatcttga

SEQ ID NO: 241 - RNA, 16 nt, Synthetic construct
  source 1..16: mol_type = other RNA
ctatccagatcttgta

SEQ ID NO: 242 - RNA, 16 nt, Synthetic construct
  source 1..16: mol_type = other RNA
atccagatcttgtgta

SEQ ID NO: 243 - RNA, 16 nt, Synthetic construct
  source 1..16: mol_type = other RNA
ccagatcttgtgtgga

SEQ ID NO: 244 - RNA, 16 nt, Synthetic construct
  source 1..16: mol_type = other RNA
ggcatctgcttcctta

SEQ ID NO: 245 - RNA, 16 nt, Synthetic construct
  source 1..16: mol_type = other RNA
gcatctgcttccttca

SEQ ID NO: 246 - RNA, 16 nt, Synthetic construct
  source 1..16: mol_type = other RNA
atctgcttccttctca

SEQ ID NO: 247 - RNA, 16 nt, Synthetic construct
  source 1..16: mol_type = other RNA
tctgcttccttctcta

SEQ ID NO: 248 - RNA, 16 nt, Synthetic construct
  source 1..16: mol_type = other RNA
tgcttccttctctcca

SEQ ID NO: 249 - RNA, 16 nt, Synthetic construct
  source 1..16: mol_type = other RNA
gcttccttctctccaa

SEQ ID NO: 250 - RNA, 16 nt, Synthetic construct
  source 1..16: mol_type = other RNA
cttctctccaaattta

SEQ ID NO: 251 - RNA, 16 nt, Synthetic construct
  source 1..16: mol_type = other RNA
ttctctccaaatttta

SEQ ID NO: 252 - RNA, 16 nt, Synthetic construct
  source 1..16: mol_type = other RNA
tctctccaaattttaa

SEQ ID NO: 253 - RNA, 16 nt, Synthetic construct
  source 1..16: mol_type = other RNA
ctctccaaattttaca

SEQ ID NO: 254 - RNA, 16 nt, Synthetic construct
  source 1..16: mol_type = other RNA
tctccaaattttacca

SEQ ID NO: 255 - RNA, 16 nt, Synthetic construct
  source 1..16: mol_type = other RNA
ctccaaattttaccca

SEQ ID NO: 256 - RNA, 16 nt, Synthetic construct
  source 1..16: mol_type = other RNA
tccaaattttacccaa

SEQ ID NO: 257 - RNA, 16 nt, Synthetic construct
  source 1..16: mol_type = other RNA
ccaaattttacccaaa

SEQ ID NO: 258 - RNA, 16 nt, Synthetic construct
  source 1..16: mol_type = other RNA
aaattttacccaagta

SEQ ID NO: 259 - RNA, 16 nt, Synthetic construct
  source 1..16: mol_type = other RNA
aattttacccaagtga

SEQ ID NO: 260 - RNA, 16 nt, Synthetic construct
  source 1..16: mol_type = other RNA
attttacccaagtgaa

SEQ ID NO: 261 - RNA, 16 nt, Synthetic construct
  source 1..16: mol_type = other RNA
tttacccaagtgacta

SEQ ID NO: 262 - RNA, 16 nt, Synthetic construct
  source 1..16: mol_type = other RNA
ttacccaagtgactta

SEQ ID NO: 263 - RNA, 16 nt, Synthetic construct
  source 1..16: mol_type = other RNA
tacccaagtgacttca

SEQ ID NO: 264 - RNA, 16 nt, Synthetic construct
  source 1..16: mol_type = other RNA
acccaagtgacttcta

SEQ ID NO: 265 - RNA, 16 nt, Synthetic construct
  source 1..16: mol_type = other RNA
cccaagtgacttctaa

SEQ ID NO: 266 - RNA, 16 nt, Synthetic construct
  source 1..16: mol_type = other RNA
ccaagtgacttctaca

SEQ ID NO: 267 - RNA, 16 nt, Synthetic construct
  source 1..16: mol_type = other RNA
caagtgacttctacaa

SEQ ID NO: 268 - RNA, 16 nt, Synthetic construct
  source 1..16: mol_type = other RNA
aagtgacttctacaca

SEQ ID NO: 269 - RNA, 16 nt, Synthetic construct
  source 1..16: mol_type = other RNA
agtgacttctacacta

SEQ ID NO: 270 - RNA, 16 nt, Synthetic construct
  source 1..16: mol_type = other RNA
tgacttctacactgta

SEQ ID NO: 271 - RNA, 16 nt, Synthetic construct
  source 1..16: mol_type = other RNA
gacttctacactgtta

SEQ ID NO: 272 - RNA, 16 nt, Synthetic construct
  source 1..16: mol_type = other RNA
acttctacactgttga

SEQ ID NO: 273 - RNA, 16 nt, Synthetic construct
  source 1..16: mol_type = other RNA
tctacactgttgaaca

SEQ ID NO: 274 - RNA, 16 nt, Synthetic construct
  source 1..16: mol_type = other RNA
ctacactgttgaacta

SEQ ID NO: 275 - RNA, 16 nt, Synthetic construct
  source 1..16: mol_type = other RNA
acactgttgaactcta

SEQ ID NO: 276 - RNA, 16 nt, Synthetic construct
  source 1..16: mol_type = other RNA
actgttgaactctgca

SEQ ID NO: 277 - RNA, 16 nt, Synthetic construct
  source 1..16: mol_type = other RNA
ctgttgaactctgcta

SEQ ID NO: 278 - RNA, 16 nt, Synthetic construct
  source 1..16: mol_type = other RNA
tgttgaactctgctta

SEQ ID NO: 279 - RNA, 16 nt, Synthetic construct
  source 1..16: mol_type = other RNA
ttgaactctgcttaca

SEQ ID NO: 280 - RNA, 16 nt, Synthetic construct
  source 1..16: mol_type = other RNA
tgaactctgcttacca

SEQ ID NO: 281 - RNA, 16 nt, Synthetic construct
  source 1..16: mol_type = other RNA
gaactctgcttaccca

SEQ ID NO: 282 - RNA, 16 nt, Synthetic construct
  source 1..16: mol_type = other RNA
aactctgcttacccaa

SEQ ID NO: 283 - RNA, 16 nt, Synthetic construct
  source 1..16: mol_type = other RNA
actctgcttacccata

SEQ ID NO: 284 - RNA, 16 nt, Synthetic construct
  source 1..16: mol_type = other RNA
tctgcttacccattca

SEQ ID NO: 285 - RNA, 16 nt, Synthetic construct
  source 1..16: mol_type = other RNA
tgcttacccattcata

SEQ ID NO: 286 - RNA, 16 nt, Synthetic construct
  source 1..16: mol_type = other RNA
gcttacccattcataa

SEQ ID NO: 287 - RNA, 16 nt, Synthetic construct
  source 1..16: mol_type = other RNA
cttacccattcataga

SEQ ID NO: 288 - RNA, 16 nt, Synthetic construct
  source 1..16: mol_type = other RNA
ttacccattcatagga

SEQ ID NO: 289 - RNA, 16 nt, Synthetic construct
  source 1..16: mol_type = other RNA
acccattcataggaca

SEQ ID NO: 290 - RNA, 16 nt, Synthetic construct
  source 1..16: mol_type = other RNA
cccattcataggacca

SEQ ID NO: 291 - RNA, 16 nt, Synthetic construct
  source 1..16: mol_type = other RNA
ccattcataggaccca

SEQ ID NO: 292 - RNA, 16 nt, Synthetic construct
  source 1..16: mol_type = other RNA
cattcataggacccta

SEQ ID NO: 293 - RNA, 16 nt, Synthetic construct
  source 1..16: mol_type = other RNA
attcataggaccctta

SEQ ID NO: 294 - RNA, 16 nt, Synthetic construct
  source 1..16: mol_type = other RNA
ttcataggacccttta

SEQ ID NO: 295 - RNA, 16 nt, Synthetic construct
  source 1..16: mol_type = other RNA
tcataggaccctttta

SEQ ID NO: 296 - RNA, 16 nt, Synthetic construct
  source 1..16: mol_type = other RNA
cataggacccttttta

SEQ ID NO: 297 - RNA, 16 nt, Synthetic construct
  source 1..16: mol_type = other RNA
gaccctttttttttaa

SEQ ID NO: 298 - RNA, 16 nt, Synthetic construct
  source 1..16: mol_type = other RNA
ccctttttttttatca

SEQ ID NO: 299 - RNA, 16 nt, Synthetic construct
  source 1..16: mol_type = other RNA
ctggctctctatcaaa

SEQ ID NO: 300 - RNA, 16 nt, Synthetic construct
  source 1..16: mol_type = other RNA
tggctctctatcaata

SEQ ID NO: 301 - RNA, 16 nt, Synthetic construct
  source 1..16: mol_type = other RNA
ccaagcttttggtgca

SEQ ID NO: 302 - RNA, 16 nt, Synthetic construct
  source 1..16: mol_type = other RNA
aagcttttggtgcata

SEQ ID NO: 303 - RNA, 16 nt, Synthetic construct
  source 1..16: mol_type = other RNA
agcttttggtgcataa

SEQ ID NO: 304 - RNA, 16 nt, Synthetic construct
  source 1..16: mol_type = other RNA
gcttttggtgcataga

SEQ ID NO: 305 - RNA, 16 nt, Synthetic construct
  source 1..16: mol_type = other RNA
cttttggtgcatagca

SEQ ID NO: 306 - RNA, 16 nt, Synthetic construct
  source 1..16: mol_type = other RNA
ttttggtgcatagcaa

SEQ ID NO: 307 - RNA, 16 nt, Synthetic construct
  source 1..16: mol_type = other RNA
tttggtgcatagcaga

SEQ ID NO: 308 - RNA, 16 nt, Synthetic construct
  source 1..16: mol_type = other RNA
ttggtgcatagcagca

SEQ ID NO: 309 - RNA, 16 nt, Synthetic construct
  source 1..16: mol_type = other RNA
tggtgcatagcagcca

SEQ ID NO: 310 - RNA, 16 nt, Synthetic construct
  source 1..16: mol_type = other RNA
gcatagcagcctggta

SEQ ID NO: 311 - RNA, 16 nt, Synthetic construct
  source 1..16: mol_type = other RNA
catagcagcctggtta

SEQ ID NO: 312 - RNA, 16 nt, Synthetic construct
  source 1..16: mol_type = other RNA
cctggttggaagcata

SEQ ID NO: 313 - RNA, 16 nt, Synthetic construct
  source 1..16: mol_type = other RNA
ctggttggaagcatta

SEQ ID NO: 314 - RNA, 16 nt, Synthetic construct
  source 1..16: mol_type = other RNA
tggttggaagcattca

SEQ ID NO: 315 - RNA, 16 nt, Synthetic construct
  source 1..16: mol_type = other RNA
ggttggaagcattcta

SEQ ID NO: 316 - RNA, 16 nt, Synthetic construct
  source 1..16: mol_type = other RNA
gttggaagcattctga

SEQ ID NO: 317 - RNA, 16 nt, Synthetic construct
  source 1..16: mol_type = other RNA
tggaagcattctgaga

SEQ ID NO: 318 - RNA, 16 nt, Synthetic construct
  source 1..16: mol_type = other RNA
ggtgggtttcattata

SEQ ID NO: 319 - RNA, 16 nt, Synthetic construct
  source 1..16: mol_type = other RNA
gtgggtttcattatca

SEQ ID NO: 320 - RNA, 16 nt, Synthetic construct
  source 1..16: mol_type = other RNA
tgggtttcattatcca

SEQ ID NO: 321 - RNA, 16 nt, Synthetic construct
  source 1..16: mol_type = other RNA
gggtttcattatccta

SEQ ID NO: 322 - RNA, 16 nt, Synthetic construct
  source 1..16: mol_type = other RNA
ggtttcattatcctga

SEQ ID NO: 323 - RNA, 16 nt, Synthetic construct
  source 1..16: mol_type = other RNA
tcattatcctgtctga

SEQ ID NO: 324 - RNA, 16 nt, Synthetic construct
  source 1..16: mol_type = other RNA
attatcctgtctgtca

SEQ ID NO: 325 - RNA, 16 nt, Synthetic construct
  source 1..16: mol_type = other RNA
gtgtgagttggacaaa

SEQ ID NO: 326 - RNA, 16 nt, Synthetic construct
  source 1..16: mol_type = other RNA
tgtgagttggacaaaa

SEQ ID NO: 327 - RNA, 16 nt, Synthetic construct
  source 1..16: mol_type = other RNA
gtgagttggacaaaaa

SEQ ID NO: 328 - RNA, 16 nt, Synthetic construct
  source 1..16: mol_type = other RNA
tgagttggacaaaaaa

SEQ ID NO: 329 - RNA, 16 nt, Synthetic construct
  source 1..16: mol_type = other RNA
gagttggacaaaaata

SEQ ID NO: 330 - RNA, 16 nt, Synthetic construct
  source 1..16: mol_type = other RNA
agttggacaaaaataa

SEQ ID NO: 331 - RNA, 16 nt, Synthetic construct
  source 1..16: mol_type = other RNA
gttggacaaaaataaa

SEQ ID NO: 332 - RNA, 16 nt, Synthetic construct
  source 1..16: mol_type = other RNA
ttggacaaaaataata

SEQ ID NO: 333 - RNA, 16 nt, Synthetic construct
  source 1..16: mol_type = other RNA
cggactgctatacaga

SEQ ID NO: 334 - RNA, 16 nt, Synthetic construct
  source 1..16: mol_type = other RNA
tgctgatttgcactca

SEQ ID NO: 335 - RNA, 16 nt, Synthetic construct
  source 1..16: mol_type = other RNA
gctgatttgcactcta

SEQ ID NO: 336 - RNA, 16 nt, Synthetic construct
  source 1..16: mol_type = other RNA
ttgcactctgctggaa

SEQ ID NO: 337 - RNA, 16 nt, Synthetic construct
  source 1..16: mol_type = other RNA
tgcactctgctggaaa

SEQ ID NO: 338 - RNA, 16 nt, Synthetic construct
  source 1..16: mol_type = other RNA
actctgctggaattca

SEQ ID NO: 339 - RNA, 16 nt, Synthetic construct
  source 1..16: mol_type = other RNA
ctctgctggaattcta

SEQ ID NO: 340 - RNA, 16 nt, Synthetic construct
  source 1..16: mol_type = other RNA
ctggaattctgcctaa

SEQ ID NO: 341 - RNA, 16 nt, Synthetic construct
  source 1..16: mol_type = other RNA
gaattctgcctagcta

SEQ ID NO: 342 - RNA, 16 nt, Synthetic construct
  source 1..16: mol_type = other RNA
aattctgcctagctga

SEQ ID NO: 343 - RNA, 16 nt, Synthetic construct
  source 1..16: mol_type = other RNA
attctgcctagctgta

SEQ ID NO: 344 - RNA, 16 nt, Synthetic construct
  source 1..16: mol_type = other RNA
ttctgcctagctgtga

SEQ ID NO: 345 - RNA, 16 nt, Synthetic construct
  source 1..16: mol_type = other RNA
gtggaaacaggcttaa

SEQ ID NO: 346 - RNA, 16 nt, Synthetic construct
  source 1..16: mol_type = other RNA
tggaaacaggcttaca

SEQ ID NO: 347 - RNA, 16 nt, Synthetic construct
  source 1..16: mol_type = other RNA
ggaaacaggcttacta

SEQ ID NO: 348 - RNA, 16 nt, Synthetic construct
  source 1..16: mol_type = other RNA
gaaacaggcttactca

SEQ ID NO: 349 - RNA, 16 nt, Synthetic construct
  source 1..16: mol_type = other RNA
acaggcttactctgaa

SEQ ID NO: 350 - RNA, 16 nt, Synthetic construct
  source 1..16: mol_type = other RNA
caggcttactctgaca

SEQ ID NO: 351 - RNA, 16 nt, Synthetic construct
  source 1..16: mol_type = other RNA
aggcttactctgacta

SEQ ID NO: 352 - RNA, 16 nt, Synthetic construct
  source 1..16: mol_type = other RNA
ggcttactctgactta

SEQ ID NO: 353 - RNA, 16 nt, Synthetic construct
  source 1..16: mol_type = other RNA
gcttactctgacttca

SEQ ID NO: 354 - RNA, 16 nt, Synthetic construct
  source 1..16: mol_type = other RNA
cttactctgacttcca

SEQ ID NO: 355 - RNA, 16 nt, Synthetic construct
  source 1..16: mol_type = other RNA
ttactctgacttccca

SEQ ID NO: 356 - RNA, 16 nt, Synthetic construct
  source 1..16: mol_type = other RNA
tactctgacttcccta

SEQ ID NO: 357 - RNA, 16 nt, Synthetic construct
  source 1..16: mol_type = other RNA
actctgacttccctga

SEQ ID NO: 358 - RNA, 16 nt, Synthetic construct
  source 1..16: mol_type = other RNA
ttttcctgcctcacaa

SEQ ID NO: 359 - RNA, 16 nt, Synthetic construct
  source 1..16: mol_type = other RNA
cctgcctcacagttaa

SEQ ID NO: 360 - RNA, 16 nt, Synthetic construct
  source 1..16: mol_type = other RNA
ctgcctcacagttaca

SEQ ID NO: 361 - RNA, 16 nt, Synthetic construct
  source 1..16: mol_type = other RNA
tgcctcacagttacaa

SEQ ID NO: 362 - RNA, 16 nt, Synthetic construct
  source 1..16: mol_type = other RNA
actattgacttcttaa

SEQ ID NO: 363 - RNA, 16 nt, Synthetic construct
  source 1..16: mol_type = other RNA
tattgacttcttaaga

SEQ ID NO: 364 - RNA, 16 nt, Synthetic construct
  source 1..16: mol_type = other RNA
attgacttcttaagaa

SEQ ID NO: 365 - RNA, 16 nt, Synthetic construct
  source 1..16: mol_type = other RNA
tcttaagaaaaaagga

SEQ ID NO: 366 - RNA, 16 nt, Synthetic construct
  source 1..16: mol_type = other RNA
cttaagaaaaaaggga

SEQ ID NO: 367 - RNA, 16 nt, Synthetic construct
  source 1..16: mol_type = other RNA
ttaagaaaaaagggaa

SEQ ID NO: 368 - RNA, 16 nt, Synthetic construct
  source 1..16: mol_type = other RNA
taagaaaaaagggaga

SEQ ID NO: 369 - RNA, 16 nt, Synthetic construct
  source 1..16: mol_type = other RNA
aagaaaaaagggagaa

SEQ ID NO: 370 - RNA, 16 nt, Synthetic construct
  source 1..16: mol_type = other RNA
gaaaaaagggagaaaa

SEQ ID NO: 371 - RNA, 16 nt, Synthetic construct
  source 1..16: mol_type = other RNA
agaaatattaatcaga

SEQ ID NO: 372 - RNA, 16 nt, Synthetic construct
  source 1..16: mol_type = other RNA
gaaatattaatcagaa

SEQ ID NO: 373 - RNA, 16 nt, Synthetic construct
  source 1..16: mol_type = other RNA
aaatattaatcagaaa

SEQ ID NO: 374 - RNA, 16 nt, Synthetic construct
  source 1..16: mol_type = other RNA
aagttgattcttatga

SEQ ID NO: 375 - RNA, 16 nt, Synthetic construct
  source 1..16: mol_type = other RNA
agttgattcttatgaa

SEQ ID NO: 376 - RNA, 16 nt, Synthetic construct
  source 1..16: mol_type = other RNA
aaagcaaagcttgaga

SEQ ID NO: 377 - RNA, 16 nt, Synthetic construct
  source 1..16: mol_type = other RNA
aagcaaagcttgagta

SEQ ID NO: 378 - RNA, 16 nt, Synthetic construct
  source 1..16: mol_type = other RNA
agcaaagcttgagtta

SEQ ID NO: 379 - RNA, 16 nt, Synthetic construct
  source 1..16: mol_type = other RNA
gcaaagcttgagttta

SEQ ID NO: 380 - RNA, 16 nt, Synthetic construct
  source 1..16: mol_type = other RNA
caaagcttgagtttca

SEQ ID NO: 381 - RNA, 16 nt, Synthetic construct
  source 1..16: mol_type = other RNA
aaagcttgagtttcca

SEQ ID NO: 382 - RNA, 16 nt, Synthetic construct
  source 1..16: mol_type = other RNA
agcttgagtttcctaa

SEQ ID NO: 383 - RNA, 16 nt, Synthetic construct
  source 1..16: mol_type = other RNA
gcttgagtttcctaaa

SEQ ID NO: 384 - RNA, 16 nt, Synthetic construct
  source 1..16: mol_type = other RNA
cttgagtttcctaaaa

SEQ ID NO: 385 - RNA, 21 nt, Synthetic construct
  source 1..21: mol_type = other RNA
tttcatcttctgaaagtcatc

SEQ ID NO: 386 - RNA, 21 nt, Synthetic construct
  source 1..21: mol_type = other RNA
tcttcatcttctgaaagtcat

SEQ ID NO: 387 - RNA, 21 nt, Synthetic construct
  source 1..21: mol_type = other RNA
tccttcatcttctgaaagtca

SEQ ID NO: 388 - RNA, 21 nt, Synthetic construct
  source 1..21: mol_type = other RNA
tacttaccttcatcttctgaa

SEQ ID NO: 389 - RNA, 21 nt, Synthetic construct
  source 1..21: mol_type = other RNA
ttacttaccttcatcttctga

SEQ ID NO: 390 - RNA, 21 nt, Synthetic construct
  source 1..21: mol_type = other RNA
tctacttaccttcatcttctg

SEQ ID NO: 391 - RNA, 21 nt, Synthetic construct
  source 1..21: mol_type = other RNA
ttctacttaccttcatcttct

SEQ ID NO: 392 - RNA, 21 nt, Synthetic construct
  source 1..21: mol_type = other RNA
tttctacttaccttcatcttc

SEQ ID NO: 393 - RNA, 21 nt, Synthetic construct
  source 1..21: mol_type = other RNA
tggtttctacttaccttcatc

SEQ ID NO: 394 - RNA, 21 nt, Synthetic construct
  source 1..21: mol_type = other RNA
tttctcagtcccatcaacggt

SEQ ID NO: 395 - RNA, 21 nt, Synthetic construct
  source 1..21: mol_type = other RNA
tgaggttttaactctggtttc

SEQ ID NO: 396 - RNA, 21 nt, Synthetic construct
  source 1..21: mol_type = other RNA
taagaggttttaactctggtt

SEQ ID NO: 397 - RNA, 21 nt, Synthetic construct
  source 1..21: mol_type = other RNA
tgagtcctcagaagctccaaa

SEQ ID NO: 398 - RNA, 21 nt, Synthetic construct
  source 1..21: mol_type = other RNA
ttgagtcctcagaagctccaa

SEQ ID NO: 399 - RNA, 21 nt, Synthetic construct
  source 1..21: mol_type = other RNA
tggtgttgccaactgtgcccg

SEQ ID NO: 400 - RNA, 21 nt, Synthetic construct
  source 1..21: mol_type = other RNA
tatggtgttgccaactgtgcc

SEQ ID NO: 401 - RNA, 21 nt, Synthetic construct
  source 1..21: mol_type = other RNA
tgatggtgttgccaactgtgc

SEQ ID NO: 402 - RNA, 21 nt, Synthetic construct
  source 1..21: mol_type = other RNA
ttgatggtgttgccaactgtg

SEQ ID NO: 403 - RNA, 21 nt, Synthetic construct
  source 1..21: mol_type = other RNA
ttcatgatggtgttgccaact

SEQ ID NO: 404 - RNA, 21 nt, Synthetic construct
  source 1..21: mol_type = other RNA
tgtcatgatggtgttgccaac

SEQ ID NO: 405 - RNA, 21 nt, Synthetic construct
  source 1..21: mol_type = other RNA
tgtgatgtcatgatggtgttg

SEQ ID NO: 406 - RNA, 21 nt, Synthetic construct
  source 1..21: mol_type = other RNA
ttgtgatgtcatgatggtgtt

SEQ ID NO: 407 - RNA, 21 nt, Synthetic construct
  source 1..21: mol_type = other RNA
tttgtgatgtcatgatggtgt

SEQ ID NO: 408 - RNA, 21 nt, Synthetic construct
  source 1..21: mol_type = other RNA
taacaggttgtgatgtcatga

SEQ ID NO: 409 - RNA, 21 nt, Synthetic construct
  source 1..21: mol_type = other RNA
tgaacaggttgtgatgtcatg

SEQ ID NO: 410 - RNA, 21 nt, Synthetic construct
  source 1..21: mol_type = other RNA
tggaacaggttgtgatgtcat

SEQ ID NO: 411 - RNA, 21 nt, Synthetic construct
  source 1..21: mol_type = other RNA
tgggaacaggttgtgatgtca

SEQ ID NO: 412 - RNA, 21 nt, Synthetic construct
  source 1..21: mol_type = other RNA
tttgggaacaggttgtgatgt

SEQ ID NO: 413 - RNA, 21 nt, Synthetic construct
  source 1..21: mol_type = other RNA
tattgggaacaggttgtgatg

SEQ ID NO: 414 - RNA, 21 nt, Synthetic construct
  source 1..21: mol_type = other RNA
ttcattgggaacaggttgtga

SEQ ID NO: 415 - RNA, 21 nt, Synthetic construct
  source 1..21: mol_type = other RNA
tctcattgggaacaggttgtg

SEQ ID NO: 416 - RNA, 21 nt, Synthetic construct
  source 1..21: mol_type = other RNA
tgtctcattgggaacaggttg

SEQ ID NO: 417 - RNA, 21 nt, Synthetic construct
  source 1..21: mol_type = other RNA
tttgatgggagcactatgatg

SEQ ID NO: 418 - RNA, 21 nt, Synthetic construct
  source 1..21: mol_type = other RNA
ttttgatgggagcactatgat

SEQ ID NO: 419 - RNA, 21 nt, Synthetic construct
  source 1..21: mol_type = other RNA
tatttgatgggagcactatga

SEQ ID NO: 420 - RNA, 21 nt, Synthetic construct
  source 1..21: mol_type = other RNA
tcatttgatgggagcactatg

SEQ ID NO: 421 - RNA, 21 nt, Synthetic construct
  source 1..21: mol_type = other RNA
ttagtcccaataactttgatt

SEQ ID NO: 422 - RNA, 21 nt, Synthetic construct
  source 1..21: mol_type = other RNA
tatagtcccaataactttgat

SEQ ID NO: 423 - RNA, 21 nt, Synthetic construct
  source 1..21: mol_type = other RNA
tgatagtcccaataactttga

SEQ ID NO: 424 - RNA, 21 nt, Synthetic construct
  source 1..21: mol_type = other RNA
tggatagtcccaataactttg

SEQ ID NO: 425 - RNA, 21 nt, Synthetic construct
  source 1..21: mol_type = other RNA
ttggatagtcccaataacttt

SEQ ID NO: 426 - RNA, 21 nt, Synthetic construct
  source 1..21: mol_type = other RNA
tctggatagtcccaataactt

SEQ ID NO: 427 - RNA, 21 nt, Synthetic construct
  source 1..21: mol_type = other RNA
ttctggatagtcccaataact

SEQ ID NO: 428 - RNA, 21 nt, Synthetic construct
  source 1..21: mol_type = other RNA
tatctggatagtcccaataac

SEQ ID NO: 429 - RNA, 21 nt, Synthetic construct
  source 1..21: mol_type = other RNA
tgatctggatagtcccaataa

SEQ ID NO: 430 - RNA, 21 nt, Synthetic construct
  source 1..21: mol_type = other RNA
tagatctggatagtcccaata

SEQ ID NO: 431 - RNA, 21 nt, Synthetic construct
  source 1..21: mol_type = other RNA
taagatctggatagtcccaat

SEQ ID NO: 432 - RNA, 21 nt, Synthetic construct
  source 1..21: mol_type = other RNA
tcaagatctggatagtcccaa

SEQ ID NO: 433 - RNA, 21 nt, Synthetic construct
  source 1..21: mol_type = other RNA
tacaagatctggatagtccca

SEQ ID NO: 434 - RNA, 21 nt, Synthetic construct
  source 1..21: mol_type = other RNA
tacacaagatctggatagtcc

SEQ ID NO: 435 - RNA, 21 nt, Synthetic construct
  source 1..21: mol_type = other RNA
tccacacaagatctggatagt

SEQ ID NO: 436 - RNA, 21 nt, Synthetic construct
  source 1..21: mol_type = other RNA
taaggaagcagatgccaaaat

SEQ ID NO: 437 - RNA, 21 nt, Synthetic construct
  source 1..21: mol_type = other RNA
tgaaggaagcagatgccaaaa

SEQ ID NO: 438 - RNA, 21 nt, Synthetic construct
  source 1..21: mol_type = other RNA
tgagaaggaagcagatgccaa

SEQ ID NO: 439 - RNA, 21 nt, Synthetic construct
  source 1..21: mol_type = other RNA
tagagaaggaagcagatgcca

SEQ ID NO: 440 - RNA, 21 nt, Synthetic construct
  source 1..21: mol_type = other RNA
tggagagaaggaagcagatgc

SEQ ID NO: 441 - RNA, 21 nt, Synthetic construct
  source 1..21: mol_type = other RNA
ttggagagaaggaagcagatg

SEQ ID NO: 442 - RNA, 21 nt, Synthetic construct
  source 1..21: mol_type = other RNA
taaatttggagagaaggaagc

SEQ ID NO: 443 - RNA, 21 nt, Synthetic construct
  source 1..21: mol_type = other RNA
taaaatttggagagaaggaag

SEQ ID NO: 444 - RNA, 21 nt, Synthetic construct
  source 1..21: mol_type = other RNA
ttaaaatttggagagaaggaa

SEQ ID NO: 445 - RNA, 21 nt, Synthetic construct
  source 1..21: mol_type = other RNA
tgtaaaatttggagagaagga

SEQ ID NO: 446 - RNA, 21 nt, Synthetic construct
  source 1..21: mol_type = other RNA
tggtaaaatttggagagaagg

SEQ ID NO: 447 - RNA, 21 nt, Synthetic construct
  source 1..21: mol_type = other RNA
tgggtaaaatttggagagaag

SEQ ID NO: 448 - RNA, 21 nt, Synthetic construct
  source 1..21: mol_type = other RNA
ttgggtaaaatttggagagaa

SEQ ID NO: 449 - RNA, 21 nt, Synthetic construct
  source 1..21: mol_type = other RNA
tttgggtaaaatttggagaga

SEQ ID NO: 450 - RNA, 21 nt, Synthetic construct
  source 1..21: mol_type = other RNA
tacttgggtaaaatttggaga

SEQ ID NO: 451 - RNA, 21 nt, Synthetic construct
  source 1..21: mol_type = other RNA
tcacttgggtaaaatttggag

SEQ ID NO: 452 - RNA, 21 nt, Synthetic construct
  source 1..21: mol_type = other RNA
ttcacttgggtaaaatttgga

SEQ ID NO: 453 - RNA, 21 nt, Synthetic construct
  source 1..21: mol_type = other RNA
tagtcacttgggtaaaatttg

SEQ ID NO: 454 - RNA, 21 nt, Synthetic construct
  source 1..21: mol_type = other RNA
taagtcacttgggtaaaattt

SEQ ID NO: 455 - RNA, 21 nt, Synthetic construct
  source 1..21: mol_type = other RNA
tgaagtcacttgggtaaaatt

SEQ ID NO: 456 - RNA, 21 nt, Synthetic construct
  source 1..21: mol_type = other RNA
tagaagtcacttgggtaaaat

SEQ ID NO: 457 - RNA, 21 nt, Synthetic construct
  source 1..21: mol_type = other RNA
ttagaagtcacttgggtaaaa

SEQ ID NO: 458 - RNA, 21 nt, Synthetic construct
  source 1..21: mol_type = other RNA
tgtagaagtcacttgggtaaa

SEQ ID NO: 459 - RNA, 21 nt, Synthetic construct
  source 1..21: mol_type = other RNA
ttgtagaagtcacttgggtaa

SEQ ID NO: 460 - RNA, 21 nt, Synthetic construct
  source 1..21: mol_type = other RNA
tgtgtagaagtcacttgggta

SEQ ID NO: 461 - RNA, 21 nt, Synthetic construct
  source 1..21: mol_type = other RNA
tagtgtagaagtcacttgggt

SEQ ID NO: 462 - RNA, 21 nt, Synthetic construct
  source 1..21: mol_type = other RNA
tacagtgtagaagtcacttgg

SEQ ID NO: 463 - RNA, 21 nt, Synthetic construct
  source 1..21: mol_type = other RNA
taacagtgtagaagtcacttg

SEQ ID NO: 464 - RNA, 21 nt, Synthetic construct
  source 1..21: mol_type = other RNA
tcaacagtgtagaagtcactt

SEQ ID NO: 465 - RNA, 21 nt, Synthetic construct
  source 1..21: mol_type = other RNA
tgttcaacagtgtagaagtca

SEQ ID NO: 466 - RNA, 21 nt, Synthetic construct
  source 1..21: mol_type = other RNA
tagttcaacagtgtagaagtc

SEQ ID NO: 467 - RNA, 21 nt, Synthetic construct
  source 1..21: mol_type = other RNA
tagagttcaacagtgtagaag

SEQ ID NO: 468 - RNA, 21 nt, Synthetic construct
  source 1..21: mol_type = other RNA
tgcagagttcaacagtgtaga

SEQ ID NO: 469 - RNA, 21 nt, Synthetic construct
  source 1..21: mol_type = other RNA
tagcagagttcaacagtgtag

SEQ ID NO: 470 - RNA, 21 nt, Synthetic construct
  source 1..21: mol_type = other RNA
taagcagagttcaacagtgta

SEQ ID NO: 471 - RNA, 21 nt, Synthetic construct
  source 1..21: mol_type = other RNA
tgtaagcagagttcaacagtg

SEQ ID NO: 472 - RNA, 21 nt, Synthetic construct
  source 1..21: mol_type = other RNA
tggtaagcagagttcaacagt

SEQ ID NO: 473 - RNA, 21 nt, Synthetic construct
  source 1..21: mol_type = other RNA
tgggtaagcagagttcaacag

SEQ ID NO: 474 - RNA, 21 nt, Synthetic construct
  source 1..21: mol_type = other RNA
ttgggtaagcagagttcaaca

SEQ ID NO: 475 - RNA, 21 nt, Synthetic construct
  source 1..21: mol_type = other RNA
tatgggtaagcagagttcaac

SEQ ID NO: 476 - RNA, 21 nt, Synthetic construct
  source 1..21: mol_type = other RNA
tgaatgggtaagcagagttca

SEQ ID NO: 477 - RNA, 21 nt, Synthetic construct
  source 1..21: mol_type = other RNA
tatgaatgggtaagcagagtt

SEQ ID NO: 478 - RNA, 21 nt, Synthetic construct
  source 1..21: mol_type = other RNA
ttatgaatgggtaagcagagt

SEQ ID NO: 479 - RNA, 21 nt, Synthetic construct
  source 1..21: mol_type = other RNA
tctatgaatgggtaagcagag

SEQ ID NO: 480 - RNA, 21 nt, Synthetic construct
  source 1..21: mol_type = other RNA
tcctatgaatgggtaagcaga

SEQ ID NO: 481 - RNA, 21 nt, Synthetic construct
  source 1..21: mol_type = other RNA
tgtcctatgaatgggtaagca

SEQ ID NO: 482 - RNA, 21 nt, Synthetic construct
  source 1..21: mol_type = other RNA
tggtcctatgaatgggtaagc

SEQ ID NO: 483 - RNA, 21 nt, Synthetic construct
  source 1..21: mol_type = other RNA
tgggtcctatgaatgggtaag

SEQ ID NO: 484 - RNA, 21 nt, Synthetic construct
  source 1..21: mol_type = other RNA
tagggtcctatgaatgggtaa

SEQ ID NO: 485 - RNA, 21 nt, Synthetic construct
  source 1..21: mol_type = other RNA
taagggtcctatgaatgggta

SEQ ID NO: 486 - RNA, 21 nt, Synthetic construct
  source 1..21: mol_type = other RNA
taaagggtcctatgaatgggt

SEQ ID NO: 487 - RNA, 21 nt, Synthetic construct
  source 1..21: mol_type = other RNA
taaaagggtcctatgaatggg

SEQ ID NO: 488 - RNA, 21 nt, Synthetic construct
  source 1..21: mol_type = other RNA
taaaaagggtcctatgaatgg

SEQ ID NO: 489 - RNA, 21 nt, Synthetic construct
  source 1..21: mol_type = other RNA
ttaaaaaaaaagggtcctatg

SEQ ID NO: 490 - RNA, 21 nt, Synthetic construct
  source 1..21: mol_type = other RNA
tgataaaaaaaaagggtccta

SEQ ID NO: 491 - RNA, 21 nt, Synthetic construct
  source 1..21: mol_type = other RNA
tttgatagagagccagagatg

SEQ ID NO: 492 - RNA, 21 nt, Synthetic construct
  source 1..21: mol_type = other RNA
tattgatagagagccagagat

SEQ ID NO: 493 - RNA, 21 nt, Synthetic construct
  source 1..21: mol_type = other RNA
tgcaccaaaagcttggttaac

SEQ ID NO: 494 - RNA, 21 nt, Synthetic construct
  source 1..21: mol_type = other RNA
tatgcaccaaaagcttggtta

SEQ ID NO: 495 - RNA, 21 nt, Synthetic construct
  source 1..21: mol_type = other RNA
ttatgcaccaaaagcttggtt

SEQ ID NO: 496 - RNA, 21 nt, Synthetic construct
  source 1..21: mol_type = other RNA
tctatgcaccaaaagcttggt

SEQ ID NO: 497 - RNA, 21 nt, Synthetic construct
  source 1..21: mol_type = other RNA
tgctatgcaccaaaagcttgg

SEQ ID NO: 498 - RNA, 21 nt, Synthetic construct
  source 1..21: mol_type = other RNA
ttgctatgcaccaaaagcttg

SEQ ID NO: 499 - RNA, 21 nt, Synthetic construct
  source 1..21: mol_type = other RNA
tctgctatgcaccaaaagctt

SEQ ID NO: 500 - RNA, 21 nt, Synthetic construct
  source 1..21: mol_type = other RNA
tgctgctatgcaccaaaagct

SEQ ID NO: 501 - RNA, 21 nt, Synthetic construct
  source 1..21: mol_type = other RNA
tggctgctatgcaccaaaagc

SEQ ID NO: 502 - RNA, 21 nt, Synthetic construct
  source 1..21: mol_type = other RNA
taccaggctgctatgcaccaa

SEQ ID NO: 503 - RNA, 21 nt, Synthetic construct
  source 1..21: mol_type = other RNA
taaccaggctgctatgcacca

SEQ ID NO: 504 - RNA, 21 nt, Synthetic construct
  source 1..21: mol_type = other RNA
tatgcttccaaccaggctgct

SEQ ID NO: 505 - RNA, 21 nt, Synthetic construct
  source 1..21: mol_type = other RNA
taatgcttccaaccaggctgc

SEQ ID NO: 506 - RNA, 21 nt, Synthetic construct
  source 1..21: mol_type = other RNA
tgaatgcttccaaccaggctg

SEQ ID NO: 507 - RNA, 21 nt, Synthetic construct
  source 1..21: mol_type = other RNA
tagaatgcttccaaccaggct

SEQ ID NO: 508 - RNA, 21 nt, Synthetic construct
  source 1..21: mol_type = other RNA
tcagaatgcttccaaccaggc

SEQ ID NO: 509 - RNA, 21 nt, Synthetic construct
  source 1..21: mol_type = other RNA
tctcagaatgcttccaaccag

SEQ ID NO: 510 - RNA, 21 nt, Synthetic construct
  source 1..21: mol_type = other RNA
tataatgaaacccaccagggc

SEQ ID NO: 511 - RNA, 21 nt, Synthetic construct
  source 1..21: mol_type = other RNA
tgataatgaaacccaccaggg

SEQ ID NO: 512 - RNA, 21 nt, Synthetic construct
  source 1..21: mol_type = other RNA
tggataatgaaacccaccagg

SEQ ID NO: 513 - RNA, 21 nt, Synthetic construct
  source 1..21: mol_type = other RNA
taggataatgaaacccaccag

SEQ ID NO: 514 - RNA, 21 nt, Synthetic construct
  source 1..21: mol_type = other RNA
tcaggataatgaaacccacca

SEQ ID NO: 515 - RNA, 21 nt, Synthetic construct
  source 1..21: mol_type = other RNA
tcagacaggataatgaaaccc

SEQ ID NO: 516 - RNA, 21 nt, Synthetic construct
  source 1..21: mol_type = other RNA
tgacagacaggataatgaaac

SEQ ID NO: 517 - RNA, 21 nt, Synthetic construct
  source 1..21: mol_type = other RNA
tttgtccaactcacactgcag

SEQ ID NO: 518 - RNA, 21 nt, Synthetic construct
  source 1..21: mol_type = other RNA
ttttgtccaactcacactgca

SEQ ID NO: 519 - RNA, 21 nt, Synthetic construct
  source 1..21: mol_type = other RNA
tttttgtccaactcacactgc

SEQ ID NO: 520 - RNA, 21 nt, Synthetic construct
  source 1..21: mol_type = other RNA
ttttttgtccaactcacactg

SEQ ID NO: 521 - RNA, 21 nt, Synthetic construct
  source 1..21: mol_type = other RNA
tatttttgtccaactcacact

SEQ ID NO: 522 - RNA, 21 nt, Synthetic construct
  source 1..21: mol_type = other RNA
ttatttttgtccaactcacac

SEQ ID NO: 523 - RNA, 21 nt, Synthetic construct
  source 1..21: mol_type = other RNA
tttatttttgtccaactcaca

SEQ ID NO: 524 - RNA, 21 nt, Synthetic construct
  source 1..21: mol_type = other RNA
tattatttttgtccaactcac

SEQ ID NO: 525 - RNA, 21 nt, Synthetic construct
  source 1..21: mol_type = other RNA
tctgtatagcagtccgtggta

SEQ ID NO: 526 - RNA, 21 nt, Synthetic construct
  source 1..21: mol_type = other RNA
tgagtgcaaatcagcatcaga

SEQ ID NO: 527 - RNA, 21 nt, Synthetic construct
  source 1..21: mol_type = other RNA
tagagtgcaaatcagcatcag

SEQ ID NO: 528 - RNA, 21 nt, Synthetic construct
  source 1..21: mol_type = other RNA
ttccagcagagtgcaaatcag

SEQ ID NO: 529 - RNA, 21 nt, Synthetic construct
  source 1..21: mol_type = other RNA
tttccagcagagtgcaaatca

SEQ ID NO: 530 - RNA, 21 nt, Synthetic construct
  source 1..21: mol_type = other RNA
tgaattccagcagagtgcaaa

SEQ ID NO: 531 - RNA, 21 nt, Synthetic construct
  source 1..21: mol_type = other RNA
tagaattccagcagagtgcaa

SEQ ID NO: 532 - RNA, 21 nt, Synthetic construct
  source 1..21: mol_type = other RNA
ttaggcagaattccagcagag

SEQ ID NO: 533 - RNA, 21 nt, Synthetic construct
  source 1..21: mol_type = other RNA
tagctaggcagaattccagca

SEQ ID NO: 534 - RNA, 21 nt, Synthetic construct
  source 1..21: mol_type = other RNA
tcagctaggcagaattccagc

SEQ ID NO: 535 - RNA, 21 nt, Synthetic construct
  source 1..21: mol_type = other RNA
tacagctaggcagaattccag

SEQ ID NO: 536 - RNA, 21 nt, Synthetic construct
  source 1..21: mol_type = other RNA
tcacagctaggcagaattcca

SEQ ID NO: 537 - RNA, 21 nt, Synthetic construct
  source 1..21: mol_type = other RNA
ttaagcctgtttccaccgcag

SEQ ID NO: 538 - RNA, 21 nt, Synthetic construct
  source 1..21: mol_type = other RNA
tgtaagcctgtttccaccgca

SEQ ID NO: 539 - RNA, 21 nt, Synthetic construct
  source 1..21: mol_type = other RNA
tagtaagcctgtttccaccgc

SEQ ID NO: 540 - RNA, 21 nt, Synthetic construct
  source 1..21: mol_type = other RNA
tgagtaagcctgtttccaccg

SEQ ID NO: 541 - RNA, 21 nt, Synthetic construct
  source 1..21: mol_type = other RNA
ttcagagtaagcctgtttcca

SEQ ID NO: 542 - RNA, 21 nt, Synthetic construct
  source 1..21: mol_type = other RNA
tgtcagagtaagcctgtttcc

SEQ ID NO: 543 - RNA, 21 nt, Synthetic construct
  source 1..21: mol_type = other RNA
tagtcagagtaagcctgtttc

SEQ ID NO: 544 - RNA, 21 nt, Synthetic construct
  source 1..21: mol_type = other RNA
taagtcagagtaagcctgttt

SEQ ID NO: 545 - RNA, 21 nt, Synthetic construct
  source 1..21: mol_type = other RNA
tgaagtcagagtaagcctgtt

SEQ ID NO: 546 - RNA, 21 nt, Synthetic construct
  source 1..21: mol_type = other RNA
tggaagtcagagtaagcctgt

SEQ ID NO: 547 - RNA, 21 nt, Synthetic construct
  source 1..21: mol_type = other RNA
tgggaagtcagagtaagcctg

SEQ ID NO: 548 - RNA, 21 nt, Synthetic construct
  source 1..21: mol_type = other RNA
tagggaagtcagagtaagcct

SEQ ID NO: 549 - RNA, 21 nt, Synthetic construct
  source 1..21: mol_type = other RNA
tcagggaagtcagagtaagcc

SEQ ID NO: 550 - RNA, 21 nt, Synthetic construct
  source 1..21: mol_type = other RNA
ttgtgaggcaggaaaagtaca

SEQ ID NO: 551 - RNA, 21 nt, Synthetic construct
  source 1..21: mol_type = other RNA
ttaactgtgaggcaggaaaag

SEQ ID NO: 552 - RNA, 21 nt, Synthetic construct
  source 1..21: mol_type = other RNA
tgtaactgtgaggcaggaaaa

SEQ ID NO: 553 - RNA, 21 nt, Synthetic construct
  source 1..21: mol_type = other RNA
ttgtaactgtgaggcaggaaa

SEQ ID NO: 554 - RNA, 21 nt, Synthetic construct
  source 1..21: mol_type = other RNA
ttaagaagtcaatagttcttc

SEQ ID NO: 555 - RNA, 21 nt, Synthetic construct
  source 1..21: mol_type = other RNA
tcttaagaagtcaatagttct

SEQ ID NO: 556 - RNA, 21 nt, Synthetic construct
  source 1..21: mol_type = other RNA
ttcttaagaagtcaatagttc

SEQ ID NO: 557 - RNA, 21 nt, Synthetic construct
  source 1..21: mol_type = other RNA
tccttttttcttaagaagtca

SEQ ID NO: 558 - RNA, 21 nt, Synthetic construct
  source 1..21: mol_type = other RNA
tcccttttttcttaagaagtc

SEQ ID NO: 559 - RNA, 21 nt, Synthetic construct
  source 1..21: mol_type = other RNA
ttcccttttttcttaagaagt

SEQ ID NO: 560 - RNA, 21 nt, Synthetic construct
  source 1..21: mol_type = other RNA
tctcccttttttcttaagaag

SEQ ID NO: 561 - RNA, 21 nt, Synthetic construct
  source 1..21: mol_type = other RNA
ttctcccttttttcttaagaa

SEQ ID NO: 562 - RNA, 21 nt, Synthetic construct
  source 1..21: mol_type = other RNA
ttttctcccttttttcttaag

SEQ ID NO: 563 - RNA, 21 nt, Synthetic construct
  source 1..21: mol_type = other RNA
tctgattaatatttctccctt

SEQ ID NO: 564 - RNA, 21 nt, Synthetic construct
  source 1..21: mol_type = other RNA
ttctgattaatatttctccct

SEQ ID NO: 565 - RNA, 21 nt, Synthetic construct
  source 1..21: mol_type = other RNA
tttctgattaatatttctccc

SEQ ID NO: 566 - RNA, 21 nt, Synthetic construct
  source 1..21: mol_type = other RNA
tcataagaatcaactttctga

SEQ ID NO: 567 - RNA, 21 nt, Synthetic construct
  source 1..21: mol_type = other RNA
ttcataagaatcaactttctg

SEQ ID NO: 568 - RNA, 21 nt, Synthetic construct
  source 1..21: mol_type = other RNA
tctcaagctttgcttttctat

SEQ ID NO: 569 - RNA, 21 nt, Synthetic construct
  source 1..21: mol_type = other RNA
tactcaagctttgcttttcta

SEQ ID NO: 570 - RNA, 21 nt, Synthetic construct
  source 1..21: mol_type = other RNA
taactcaagctttgcttttct

SEQ ID NO: 571 - RNA, 21 nt, Synthetic construct
  source 1..21: mol_type = other RNA
taaactcaagctttgcttttc

SEQ ID NO: 572 - RNA, 21 nt, Synthetic construct
  source 1..21: mol_type = other RNA
tgaaactcaagctttgctttt

SEQ ID NO: 573 - RNA, 21 nt, Synthetic construct
  source 1..21: mol_type = other RNA
tggaaactcaagctttgcttt

SEQ ID NO: 574 - RNA, 21 nt, Synthetic construct
  source 1..21: mol_type = other RNA
ttaggaaactcaagctttgct

SEQ ID NO: 575 - RNA, 21 nt, Synthetic construct
  source 1..21: mol_type = other RNA
tttaggaaactcaagctttgc

SEQ ID NO: 576 - RNA, 21 nt, Synthetic construct
  source 1..21: mol_type = other RNA
ttttaggaaactcaagctttg